(19) United States
(12) Patent Application Publication
(10) Pub. No.: US 2021/0137854 A1
(43) Pub. Date: May 13, 2021

(54) TAAR Receptor Agonists for the Treatment of Alopecia

(71) Applicant: APPLIED BIOLOGY, INC., Irvine, CA (US)

(72) Inventors: Ofer A. Goren, Irvine, CA (US); John McCoy, Irvine, CA (US)

(21) Appl. No.: 17/155,865

(22) Filed: Jan. 22, 2021

Related U.S. Application Data
(63) Continuation of application No. PCT/US2019/023148, filed on Mar. 20, 2019.
(60) Provisional application No. 62/703,547, filed on Jul. 26, 2018.
(60) Provisional application No. 62/711,236, filed on Jul. 27, 2018.
(60) Provisional application No. 62/711,162, filed on Jul. 27, 2018.
(60) Provisional application No. 62/733,962, filed on Sep. 20, 2018.
(60) Provisional application No. 62/752,608, filed on Oct. 30, 2018.

(51) Int. Cl. A61K 31/137; A61K 31/517; A61P 17/14; A61K 9/00; A61K 9/08

(52) U.S. Cl. CPC A61K 31/137; A61K 31/517; A61K 9/08; A61K 9/0014; A61P 17/14

(57) ABSTRACT

Hair shedding and other disorders related to mechanical pulling on hair are treated or prevented by administering a topical composition comprising a trace amine associated receptor agonist or another compound to contract the arrector pili muscle.

### CROSS-REFERENCE TO RELATED APPLICATIONS

This application relates to and claims the benefit of priority of U.S. 62/703,547 filed on Jul. 26, 2018, U.S. 62/711,162 filed on Jul. 27, 2018, U.S. 62/711,236 filed on Jul. 27, 2018, U.S. 62/733,962 filed on Sep. 20, 2018, and U.S. 62/752,608 filed on Oct. 30, 2018, the contents of each being incorporated herein by reference in its entirety.

The present application also claims priority to International Application No. PCT/US2019/023148, which claims priority to U.S. 62/703,547 filed on Jul. 26, 2018, U.S. 62/711,162 filed on Jul. 27, 2018, U.S. 62/711,236 filed on Jul. 27, 2018, U.S. 62/733,962 filed on Sep. 20, 2018, and U.S. 62/752,608 filed on Oct. 30, 2018. The entirety of PCT/US2019/023148, which published as WO 2020/023084, is incorporated by reference herein.

### FIELD

The present invention relates to methods and compositions that effect trace amine associated receptors (TAAR) to affect smooth muscle of the arrector pilli muscle located in the hair follicle. Topical compositions of TAAR binding molecules are described, as well as, methods for changing the appearance of hair or treating disorders of hair bearing skin. The present invention is directed to methods for treating, reducing or preventing alopecia and other hair loss disorders caused by mechanical pulling of the hair, including but not necessarily limited to hair shedding, and compositions, devices and kits useful in such methods.

### BACKGROUND

Trace amine-associated receptors (TAARs), sometimes referred to as trace amine receptors (TAs or TARs), are a class of G protein-coupled receptors found in mammals. TAARs bind trace amines found naturally in mammals, e.g., phenylethylamine, tyramine, and tryptamine; metabolic derivatives of the amino acids phenylalanine, tyrosine and tryptophan, respectively. TAARs also bind many synthetic compounds, e.g., ephedrine, amphetamine, methamphetamine, methylenedioxymethamphetamine (MDMA, ecstasy). Additionally, it has been shown that mammalia TAAR1 is also a receptor for thyronamines, decarboxylated and deiodinated relatives of thyroid hormones. TAAR2-TAAR9 have been shown to function as olfactory receptors for volatile amine odorants in vertebrates.

### SUMMARY

Compositions and methods are disclosed herein for changing the appearance and treating disorders of hair bearing skin. Such disorders may be treated or prevented by the application to the skin, hair follicle, or scalp of a compound or agent that induces contraction of the arrector pili (AP) muscle, such as, without limitation, a trace amine-associated receptor (TAAR) agonist or antagonist, (TAAR agonists).

TAARs are located in smooth muscle and can be stimulated to cause smooth muscle tone changes independent of the sympathetic mechanism, e.g., alpha and beta adrenergic receptors. This new mechanism provides new avenues for methods to simulate smooth muscle modulation to augment existing sympathomimetic signaling molecules. TAARs may be used to stimulate the arrector pili muscle, which is also a smooth muscle in the skin. Topical Formulations of TAAR receptors would be advantageous for application to hair bearing skin. Smooth muscle regulates the pilomoter response, i.e., “goosebumps” via the arrector pili muscle.

Exciting the pilomoter response, i.e., “goosebumps” via the arrector pili muscle can be used for treating several medical conditions. For example, exciting the pilomoter response on the scalp can be used to reduce hair shedding, treat hair loss, treat alopecia, treat traction alopecia. Additionally, exciting the pilomoter response on the scalp can be used to increase the amount of force required to pluck or remove a hair. Exciting the pilomoter response via TAAR receptors may be used for several cosmetic applications, as well. For example, increasing the force required to remove hair when brushing, stopping hair fall, and increasing the body (“fullness”) of hair.

In an exemplary embodiment, a method for treatment or reduction of hair shedding involves applying a composition comprising a pilomotor effective amount of a trace amine associated receptor agonist topically to a portion of skin on the head that includes at least one hair follicle.

In some embodiments, the at least one hair follicle is under tension.

In some embodiments, the portion of skin is at risk for developing traction alopecia.

In some embodiments, the composition comprises a trace amine associated receptor agonist that is octopamine, p-octopamine, m-octopamine, and/or a pharmaceutically acceptable salt or hydrate thereof, wherein the octopamine is present in the composition in a concentration of 1% to 60% by weight.

In some embodiments, the composition comprises the 1-enantiomer of octopamine that is R-(−)-4-(2-amino-1-hydroxyethyl)phenol and has less than 10% by weight of other enantiomers of octopamine.

In some embodiments, R-(−)-4-(2-amino-1-hydroxyethyl)phenol is present in the composition at 5% to 40% by weight.

In some embodiments, the trace amine associated receptor agonist is any one or combination of citrus aurantium (e.g. bitter orange extract), 2-phenylethylamine, p-tyramine, m-tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamyl amine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO5166017, RO5203648, RO5256390, RO5263397, and RO5212773 (EPPTB).

In some embodiments, a method for treatment or reduction of hair shedding involves applying a composition comprising a pilomotor effective amount of a trace amine associated receptor agonist and an alpha 1 adrenergic receptor agonist topically to a portion of skin on the head that includes at least one hair follicle.

In some embodiments: the alpha 1 adrenergic receptor agonist is any one or combination of cirazoline, desvenlafaxine, etilfrine, metaraminol, methoxamine, naphazoline, oxymetazoline, pseudoephrine, m-synephrine, p-synephrine, synephrine, octopamine, hordenine, tetrahydrozoline, isometheptene, metaraminol, nicergoline, ergonovine, levonordefrin, phendimetrazine, methoxamine, midodrine, clonidine, pergolide, xylometazoline, droxidopa, epinephrine, mephentermine, 4-methoxyamphetamine, Benzphetamine, Naphazoline, Apraclondine, Bromocriptine, Oxymetazoline, Phenylpropanolamine, Pseudoephedrine, Dipivefrin, and xylometazoline.

In an exemplary embodiment, a method for treatment or reduction of hair shedding involves applying a composition comprising a pilomotor effective amount of a trace amine associated receptor agonist and an alpha 1 adrenergic receptor antagonist topically to a portion of skin on the head that includes at least one hair follicle.

In some embodiments: the alpha 1 adrenergic receptor antagonist is any one or combination of (+)Dobutamine, abanoquil, Acebutolol, adimolol, ajmalicine, alfuzosin, anisodamine, Atenolol, benoxathian, Betaxolol, Bretylium, Buflomedil, Butoxamine, Carteolol, carvedilol, cirazoline, corynanthine, dihydroergocornine, dihydroergocristine, dihydroergocryptine, dihydroergotoxine, doxazosin, ergot derivatives, Esmolol, Guanadrel, Guanethidine, hydroxymaprotiline, ifenprodil, indoramin, ketanserin, lab etalol, Levobunolol, Metoprolol, monatepil, Moxisylyte, Nadolol, nantenine, Nicergoline, oxaprotiline, pelanserin, Penbutolol, phendioxan, phenoxybenzamine, phentolamine, Pindolol, prazosin, Propanolol, pukateine, Raubasine, rauwolscine, Reserpine, silodosin, tamsulosin, terazosin, thiamenidine, tiamenidine, Timolol, Tolazoline, umespirone, urapidil, urapidil, WB-4101, yohimbine, ziprasidone, zuclopenthixol, L-765,314, Z-350, SR 59230A, and BMY-7,378.

In an exemplary embodiment, a method of reducing hair shedding during brushing, combing or showering, involves applying a composition comprising a pilomotor effective amount of a trace amine associated receptor agonist topically to a portion of skin on the head that includes at least one hair follicle.

In some embodiments, the composition is applied to the skin prior to brushing or combing.

In some embodiments, the trace amine associated receptor agonist is octopamine and/or a pharmaceutically acceptable salt or hydrate thereof, wherein the octopamine is present in the composition in a concentration of 1% to 60% by weight.

In some embodiments, R-(−)-4-(2-amino-1-hydroxyethyl)phenol is present in the composition at 10% to 40% by weight.

In an exemplary embodiment, a method of reducing hair shedding during brushing, combing or showering involves applying a composition comprising a pilomotor effective amount of a trace amine associated receptor agonist and an alpha 1 adrenergic receptor agonist topically to a portion of skin on the head that includes at least one hair follicle.

In an exemplary embodiment, a method of reducing hair shedding during brushing, combing or showering involves applying a composition comprising a pilomotor effective amount of a trace amine associated receptor agonist and an alpha 1 adrenergic receptor antagonist topically to a portion of skin on the head that includes at least one hair follicle.

In an exemplary embodiment, a method of increasing hair epilation force threshold, the method comprising applying a composition comprising a pilomotor effective amount of a trace amine associated receptor agonist topically to a portion of skin on the head of a person that includes at least one hair follicle.

In some embodiments, before, during, and/or after the composition is applied, the person undergoes a cosmetic procedure to the hair selected from the group consisting of braiding, flat ironing, attaching a hair weave, attaching a hair extension, or tying the hair back in a ponytail.

In some embodiments, the composition is applied to the skin at a rate of once every 24 hours.

In some embodiments, the composition is applied to the skin at a rate of twice every 24 hours.

In an exemplary embodiment, a method of increasing hair epilation force threshold, the method comprising applying a composition comprising a pilomotor effective amount of a trace amine associated receptor agonist and an alpha 1 adrenergic receptor agonist topically to a portion of skin on the head of a person that includes at least one hair follicle.

In some embodiments, before, during, and/or after the composition is applied, the person undergoes a cosmetic procedure to the hair selected from the group consisting of braiding, flat ironing, attaching a hair weave, attaching a hair extension, or tying the hair back in a ponytail.

In an exemplary embodiment, a method of increasing hair epilation force threshold involves applying a composition comprising a pilomotor effective amount of a trace amine associated receptor agonist and an alpha 1 adrenergic receptor antagonist topically to a portion of skin on the head of a person that includes at least one hair follicle.

In some embodiments, before, during, and/or after the composition is applied, the person undergoes a cosmetic procedure to the hair selected from the group consisting of braiding, flat ironing, attaching a hair weave, attaching a hair extension, or tying the hair back in a ponytail.

In an exemplary embodiment, a method for prevention of traction alopecia involves applying a composition comprising a pilomoter effective amount of a trace amine associated receptor agonist to the scalp to an area with a group of follicles that will experience a pulling force.

In an exemplary embodiment, a method for prevention of traction alopecia involves applying a composition comprising a pilomoter effective amount of a combination of a trace amine associated receptor agonist and an alpha 1 adrenergic receptor agonist to the scalp to an area with a group of follicles that will experience a pulling force.

In an exemplary embodiment, a method for prevention of traction alopecia, the method comprising applying a composition comprising a pilomoter effective amount of a combination of a trace amine associated receptor agonist and an alpha 1 adrenergic receptor antagonist to the scalp to an area with a group of follicles that will experience a pulling force.

In an exemplary embodiment, a composition includes a pilomotor effective amount of a trace amine associated receptor agonist and a cosmetic hair product.

In some embodiments, the hair product comprises pre-shampoo tonic, shampoo, hair color, hair dye, hair oil, hair conditioner, hairspray, and/or hair detangling solution.

In some embodiments, a cosmetic kit includes a container containing an embodiment of the composition, the container including a pump spray bottle.

In an exemplary embodiment, a composition includes a pilomotor effective amount of a trace amine associated receptor agonist, an alpha 1 adrenergic receptor agonist, and a cosmetic hair product.

In an exemplary embodiment, a cosmetic composition includes a concentration of a trace amine that increases the epilatory force threshold of at least one hair follicle of the scalp when the cosmetic composition is applied to the scalp.

In an exemplary embodiment, a method of reducing or preventing hair shedding from brushing the hair involves applying an embodiment of the cosmetic composition to the scalp before, during, and/or after brushing.

In an exemplary embodiment, a cosmetic composition includes a concentration of a trace amine and an alpha 1 adrenergic receptor agonist that increases the epilatory force threshold of at least one hair follicle of the scalp when the cosmetic composition is applied to the scalp.

In an exemplary embodiment, a cosmetic composition includes a concentration of a trace amine and an alpha 1 adrenergic receptor antagonist that increases the epilatory force threshold of at least one hair follicle of the scalp when the cosmetic composition is applied to the scalp.

In an exemplary embodiment, a composition includes a pilomotor effective amount of a trace amine associated receptor agonist and an alpha 1 adrenergic receptor antagonist.

In an exemplary embodiment, a method of causing contraction of an arrector pili muscle involves application of an embodiment of the composition to a portion of a patient's skin, wherein contraction of the arrector pili muscle occurs by the trace amine associated receptor agonist only.

In an exemplary embodiment, a method for treatment or reduction of hair shedding or hair loss involves applying a composition comprising a pilomotor effective amount of a trace amine associated receptor agonist topically to a portion of skin on the head that includes at least one hair follicle, wherein the at least one hair follicle has, is, or will be experiencing mechanical forces from a ponytail style hair.

In an exemplary embodiment, a method for treatment or reduction of hair shedding or hair loss involves applying a composition comprising a pilomotor effective amount of a trace amine associated receptor agonist and an alpha 1 adrenergic receptor agonist topically to a portion of skin on the head that includes at least one hair follicle, wherein the at least one hair follicle has, is, or will be experiencing mechanical forces from a ponytail style hair.

In an exemplary embodiment, a method for treatment or reduction of hair shedding or hair loss involves applying a composition comprising a pilomotor effective amount of a trace amine associated receptor agonist and an alpha 1 adrenergic receptor antagonist topically to a portion of skin on the head that includes at least one hair follicle, wherein the at least one hair follicle has, is, or will be experiencing mechanical forces from a ponytail style hair.

In an exemplary embodiment, a method for treatment of alopecia involves administering a therapeutically-effective concentration of a probiotic, a genetic modified bacteria, and/or a viral vector to cause local floral to generate a trace amine.

In some embodiments, the composition comprises a trace amine associated receptor agonist that is tyramine, tyramine HCl, and/or a pharmaceutically acceptable salt or hydrate thereof.

In some embodiments, the composition comprises a trace amine associated receptor agonist that is tyramine.

In an exemplary embodiment, a method for treatment or reduction of hair shedding or hair loss involves applying a composition comprising a pilomotor effective amount of a trace amine associated receptor agonist topically to a portion of skin on the head that includes at least one hair follicle located at or near the frontal hair line of the head, wherein the at least one hair follicle has, is, or will be experiencing mechanical forces from a ponytail style hair.

In some embodiments, the method also involves treating frontal pattern traction alopecia caused by the ponytail style hair.

In some embodiments, the composition is a cosmetic composition.

In an exemplary embodiment, a method for treatment or reduction of hair shedding or hair loss involves applying a composition comprising a pilomotor effective amount of a trace amine associated receptor agonist and an alpha 1 adrenergic receptor agonist topically to a portion of skin on the head that includes at least one hair follicle located at or near the frontal hair line of the head, wherein the at least one hair follicle has, is, or will be experiencing mechanical forces from a ponytail style hair.

In some embodiments, the method also involves treating frontal pattern traction alopecia caused by the ponytail style hair.

In an exemplary embodiment, a method for treatment or reduction of hair shedding or hair loss involves applying a composition comprising a pilomotor effective amount of a trace amine associated receptor agonist and an alpha 1 adrenergic receptor antagonist topically to a portion of skin on the head that includes at least one hair follicle located at or near the frontal hair line of the head, wherein the at least one hair follicle has, is, or will be experiencing mechanical forces from a ponytail style hair.

In an exemplary embodiment, a method for preventing traction alopecia, hair loss, and/or follicular damage involves applying a composition comprising a pilomotor effective amount of a trace amine associated receptor agonist topically to a portion of skin on the head to reduce or prevent frontal pattern traction alopecia.

In some embodiments, the frontal pattern traction alopecia is caused, at least in part, by a ponytail style hair.

In an exemplary embodiment, a method for preventing traction alopecia, hair loss, and/or follicular damage involves applying a composition comprising a pilomotor effective amount of a trace amine associated receptor agonist and an alpha 1 adrenergic receptor agonist topically to a portion of skin on the head to reduce or prevent frontal pattern traction alopecia.

In an exemplary embodiment, a method for preventing traction alopecia, hair loss, and/or follicular damage involves applying a composition comprising a pilomotor effective amount of a trace amine associated receptor agonist and an alpha 1 adrenergic receptor antagonist topically to a portion of skin on the head to reduce or prevent frontal pattern traction alopecia.

In an exemplary embodiment, a method for preventing traction alopecia, hair loss, and/or follicular damage involves applying a composition comprising a pilomotor effective amount of a trace amine associated receptor agonist topically to a portion of skin on the head that includes at least one hair follicle located at or near the frontal hair line of the head, wherein the at least one hair follicle has, is, or will be experiencing mechanical forces from a ponytail style hair.

In an exemplary embodiment, a composition includes a pilomotor effective amount of a trace amine associated receptor agonist, the composition being configured as a pre-shampoo tonic, shampoo, hair color, hair dye, hair oil, hair conditioner, hairspray, hair detangling solution, moisturizing lotion, a facial cream, a sunscreen, a gel, an ointment, a foam, and/or a spray.

In some embodiments, the tyramine, tyramine HCl, and/or a pharmaceutically acceptable salt or hydrate thereof is present in the composition in a concentration of 5% to 20% by weight.

In some embodiments, the tyramine, tyramine HCl, and/or a pharmaceutically acceptable salt or hydrate thereof is present in the composition in a concentration of 1% to 10% by weight.

In some embodiments, the tyramine, tyramine HCl, and/or a pharmaceutically acceptable salt or hydrate thereof is present in the composition in a concentration of 10% to 30% by weight.

In an exemplary embodiment, a method for preventing traction alopecia, hair loss, and/or follicular damage involves applying an embodiment of the composition to a portion of skin on the head and/or face that includes at least one hair follicle.

In some embodiments, the portion of skin includes being applied at or near at least a portion of the frontal hair line of the head.

In some embodiments, the at least one hair follicle has, is, or will be experiencing mechanical forces from a ponytail style hair.

In some embodiments, the composition is allowed to sit or be absorbed into the skin after being applied.

In some embodiments, the composition is massaged into the skin after being applied.

In some embodiments, the composition is combed or brushed into the skin after being applied.

In some embodiments, the composition comprises 15% tyramine HCl solution to 20% tyramine HCl solution by weight.

### BRIEF DESCRIPTION OF THE DRAWINGS

The accompanying drawings exemplify embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1a depicts a cross sectional view of the hair follicle and the arrector pili muscle in a relaxed state; and

FIG. 1b depicts a cross sectional view of the hair follicle and the arrector pili muscle in a tensed state.

FIGS. 2 and 3 depict hair loss from mechanical pulling according to the experiments reported herein.

FIGS. 4 and 5 depict epilatory force thresholds on scalp hair follicles following topical phenylephrine application according to the procedures described in the examples reported herein.

### DETAILED DESCRIPTION

Trace amines are biologically active amines occurring in the body in trace amounts. Some examples of trace amines include tyramine, beta phenylethylamine, tryptamine and octopamine. They are structurally and functionally related to the catecholamines and there are a large number of synthetic analogues, such as, amphetamines. Endogenous trace amines are synthesized in the body by the decarboxylation of their respective precursor amino acids. Oral administration of trace amines in humans cause increase in blood pressure and trace amines supplied to isolated blood vessels cause vasoconstriction. Additionally, trace amines are theorized to affect the arrector pili muscle.

Each hair follicle in the scalp contains an arrector pili muscle that, when contracted, erects the hair. The smooth muscle in the arrector pili expresses trace amine associated receptors (“TAAR”). Disclosed herein are methods for the treatment and prevention of disorders associated with mechanical stress or pulling on the hair comprising topical administration to the scalp or hair follicle of a composition comprising one or more TAAR agonists. As shown herein, such agonists protect against hair loss as shown by an increase in epilation force needed to remove a hair and reduction in the number of hairs removed after brushing. Without intending to be limited or bound by theory, Applicants postulate that contraction of the arrector pili muscle via a TAAR agonist increases the threshold of force required to pluck hair during cosmetic procedures and while under mechanical stress. Thus, it is believed that the compounds and agents used in the present invention stimulate contraction of the AP muscle and thereby reduce hair loss by increasing the force required to remove the hair.

While the disclosure most often specifically refers to TAAR agonists as agents useful for treating and preventing the disorders described herein relating to hair loss, it should be understood that any agent that stimulates contraction of smooth muscle, and particularly the AP muscle, can be useful in the compositions and methods described herein. That is, unless specifically indicated otherwise, disclosure relating to uses or formulations of TAAR agonists should be considered to refer as well to other agents that stimulate AP muscle contraction.

As used herein, the term “traction alopecia” means a form of alopecia (hair loss or hair shedding) associated with mechanical forces that pull the hair such as hair brushing hair combing, flat ironing, wearing of extensions, hair braiding, and ponytail style hair. This can include mechanical forces that pull on hair at or near the frontal hair line as a result of brushing hair, combing hair, wearing ponytail style hair, etc. Under this definition, although chronic traction on the hair can lead to traction alopecia, the mechanical forces that pull the hair do not necessarily need to be chronic to lead to hair loss.

As used herein, the term “pilomotor effective” refers to an agent or treatment that stimulates contraction of the arrector pili muscle associated with a hair follicle. A “pilomotor effective amount” of an agent or treatment is an amount sufficient to stimulate contraction of the arrector pili muscle.

As used herein, the term “trace amine associated receptor agonist” refers to a ligand that binds the trace amine associated receptor on smooth muscle cells and activates smooth muscle contraction.

As used herein, the terms “prevent” or “prevention” and other derivatives of the words, when used in reference to alopecia, e.g., traction alopecia, refer to a reduced likelihood of alopecia in an individual receiving a given treatment relative to that of a similar individual at risk for alopecia but not receiving that treatment. As such, the terms “prevent” and “prevention” encompass a treatment that results in a lesser degree of alopecia, e.g., traction alopecia, than would be otherwise expected for a given individual. Efficacy for prevention of alopecia, e.g., traction alopecia, can be established through controlled studies, e.g., in which a subject is administered a treatment (e.g., a topical treatment) at one site likely to experience or exhibit alopecia (e.g., for traction alopecia, a site at which hair is pulled for an extended period of time) but not at another site subjected to the same conditions. Under these circumstances, if the site treated with the topical treatment undergoes less hair loss over time relative to the untreated site, e.g., at least 5% less, at least 10% less, at least 15% less, at least 20% less, at least 25% less, at least 30% less, at least 35% less, at least 40% less, at least 45% less, at least 50% less or beyond, the treatment is effective for the prevention of alopecia, e.g., traction alopecia. Efficacy for the prevention of other forms of alopecia can be established in a similar manner, e.g., by treating one area affected by or likely to be affected by such alopecia, but not another, substantially similar area (i.e., subject to the same conditions causing alopecia or a likelihood of alopecia) and comparing hair loss or retention in the two areas.

As used herein, the terms “treat,” “treatment,” or “treating” refer to therapeutic treatments, wherein the object is to reverse, alleviate, ameliorate, inhibit, slow down or stop the progression or severity of a disease or condition, e.g., traction alopecia or other form of alopecia. The term “treating” includes reducing or alleviating at least one adverse effect or symptom of a disease or condition, e.g., traction alopecia or other form of alopecia. Treatment is generally “effective” if one or more symptoms are reduced. Alternatively, treatment is “effective” if the progression of a disease is reduced or halted. That is, “treatment” includes not just the improvement of symptoms, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the extent or amount of hair loss is reduced, or the progression of hair loss is slowed or halted. The term “treatment” of a disease also includes providing relief from the symptoms or side-effects of the disease (including palliative treatment).

As used herein, the term “epilatory” relates to the removal of hair. As used herein, the term “increasing epilatory force” refers to any treatment that increases the physical force required to remove a hair. As noted, the increase in force can be viewed as at least a partial balancing of a traction force by the force exerted by the arrector pili muscle—the vector direction of the arrector pili muscle's force of contraction need not necessarily be directly opposed to a traction force on the hair shaft to increase the epilatory force required to remove the hair, but the net effect is that the muscle provides at least a partial counter-acting force to the traction force, whether it directly pulls back on the hair or simply holds the hair or hair follicle more tightly in place. An increase in epilatory force can be measured in several ways, including empirically, through a reduction in traction alopecia (e.g., 10% or less reduction in hair loss) despite continued or ongoing traction, or through measurement of actual force exerted on the hair follicle, e.g., with a myograph, trichotilometer, or a device used to measure tensile forces.

As used herein the term “comprising” or “comprises” is used in reference to compositions, methods, etc. refers to component(s) or method steps that are present in the method or composition, yet allows for the composition, method, etc. to also include unspecified elements.

The term “consisting of” refers to compositions, methods, and respective components thereof as described herein, which are exclusive of any element not recited in that description of the embodiment.

The term “piloerecting” refers to any treatment that causes the hair to stand up, i.e., causes “goosebumps” or increases the physical force required to remove a hair. Compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In various aspects, the technology described herein relates to the prevention of hair shedding and traction alopecia. One preventive approach currently available for traction alopecia is to remove, limit or avoid the application of a traction force to the hair. Thus, hairstyles or other factors that pull on the hair (e.g., tight fitting helmets) should normally be avoided to prevent traction alopecia. However, by using the approaches set out herein, such as the application of a TAAR agonist to the hair follicle or scalp, one can limit, reduce or prevent as that term is defined herein the traction alopecia-inducing effects of such hairstyles or factors despite the ongoing traction involved. This preventive approach permits one to wear a hairstyle, helmet, etc., that would normally have a high risk of inducing traction alopecia without actually suffering the traction-related hair loss.

Various aspects of the technology described herein involve pilomotor stimulation. The measurement or detection of pilomotor stimulation can be performed, at its simplest, by observation of the area at the base of the hair shaft—an agent or treatment that induces arrector pili contraction causes the hair follicle to “stand up” and causes puckering of the skin around the hair shaft commonly referred to as “goose bumps.” Thus, if an agent is applied and the hair stands up, goose bumps form, or both, the agent has stimulated the arrector pili.

Measurement of the strength of arrector pili muscle contraction can be performed, if necessary, via myograph adapted for that purpose. Examples are described in, e.g., Zeveke & Gladysheva, Bull. Exp. Biol. Med. 71: 102-105 (1971) and Hellmann, J. Physiol. 169: 603-620 (1963), each of which is incorporated herein in its entirety by reference. Other systems to measure the strength of the arrector pili muscle can use a trichotillometer or a device used to measure tensile forces. Traction alopecia is a form of alopecia, or gradual hair loss, caused primarily by pulling force applied to the hair. Several different hair styles and hair extensions can cause or exacerbate traction alopecia. For example, certain styles or braiding patterns that pull the hairline have been shown to cause traction alopecia. Particularly tight braids, barrettes, or the installation of hair extensions can exert sufficient chronic force on the hair follicles to cause traction alopecia. Generally, traction alopecia has a mechanical origin based on the force on the hair. For example, chronic pulling on the hair follicles can cause inflammation. Eventually, follicular scarring and permanent alopecia can occur from prolonged pulling.

Accordingly, the mechanical strain of the pulling force on the root causes the damage to the follicle in the root. Additionally, as illustrated in FIGS. 1A-1B, each follicular unit contains a smooth muscle anchoring the hair to the epidermis. When the smooth muscle is relaxed as illustrated in FIG. 1A, the muscle does not supply much restraining force and the follicle can be removed easily. When the smooth muscle or arrector pili (AP) contracts as illustrated in FIG. 1B, the follicle stands up and is restrained by additional force from the smooth muscle rather than just primarily the surrounding connective tissue of the dermis. Accordingly, the smooth muscle can provide more retention force in opposition to a force that would pull on the hair to dislodge the follicle if it is contracted. Thus, by contracting the arrector pili (AP) muscle, the root can be more firmly grounded into the dermis of the skin preventing the mechanical strain from damaging the root and dermis, i.e. requiring a larger epilation force for removal of the hair follicle. This would prevent the chronic stressing from pulling of the hair observed in different hairstyles from doing as much damage to the root, and thereby would prevent or reduce the risk of developing traction alopecia.

In some aspects, then, the technology described herein relates to the reduction of the force exerted on the root of a hair. In practice, this “reduction” in force is more akin to providing a better balancing force against a traction on the hair itself—that is, the treatments described herein will not necessarily reduce the amount of traction on the hair, but by stimulating the contraction of the arrector pili muscles, the treatments provide a force that at least partially counters the effect of the traction or pulling force, thereby protecting the root against the epilatory effect of the traction.

Accordingly, disclosed herein are methods for contracting the smooth muscle cells or arrector pili while a patient is wearing a hair extension, wig, a tightly woven or pulling hairstyle, combing their hair, or engaging in other behavior that pulls back on the follicles of the hair. Several methods are disclosed for contracting the AP muscle including application of composition containing a TAAR agonist.

Disorders to be Treated or Prevented

Applicants disclose herein methods to treat or prevent various conditions related to mechanical stress on the human hair. In one embodiment, the invention concerns treating, reducing or preventing hair loss from disorders such as traction alopecia, androgenic alopecia (also known as androgenetic alopecia), alopecia areata, and alopecia universalis, and hair loss due to hair brushing, combing, etc. comprising topical administration to a person in need thereof of a therapeutically effective amount of a TAAR agonist. In another embodiment, the invention concerns a method for the reduction of the force exerted on a root of a hair comprising topical administration to a person in need thereof of a therapeutically effective amount of a TAAR agonist. In another embodiment, the invention concerns a method for increasing hair epilation force comprising topical administration to a person in need thereof of a therapeutically effective amount of a TAAR agonist. In another embodiment, the invention concerns a cosmetic method for piloerecting hair or raising hair comprising topical administration to a person in need thereof of a therapeutically effective amount of a TAAR agonist. In another embodiment, the invention concerns a cosmetic method for piloerecting hair or raising hair to increase the appearance of “volume” of hair comprising topical administration to a person in need thereof of a therapeutically effective amount of a TAAR agonist.

In one embodiment, the invention concerns treating, reducing or preventing hair loss from disorders such as traction alopecia, androgenic alopecia (also known as androgenetic alopecia), alopecia areata, and alopecia universalis, and hair loss due to hair brushing, combing, etc. comprising topical administration to a person in need thereof of a therapeutically effective amount of a combination of a TAAR agonist and an adrenergic receptor agonist. In another embodiment, the invention concerns a method for the reduction of the force exerted on a root of a hair comprising topical administration to a person in need thereof of a therapeutically effective amount of a combination of a TAAR agonist and an adrenergic receptor agonist. In another embodiment, the invention concerns a method for increasing hair epilation force comprising topical administration to a person in need thereof of a therapeutically effective amount of a combination of a TAAR agonist and an adrenergic receptor agonist. In another embodiment, the invention concerns a cosmetic method for piloerecting hair or raising hair comprising topical administration to a person in need thereof of a therapeutically effective amount of a combination of a TAAR agonist and an adrenergic receptor agonist. In another embodiment, the invention concerns a cosmetic method for piloerecting hair or raising hair to increase the appearance of “volume” of hair comprising topical administration to a person in need thereof of a therapeutically effective amount of a combination of a TAAR agonist and an adrenergic receptor agonist.

In one aspect, the therapeutically effective amount of the agent administered, such as the TAAR agonist, is a pilomotor effective amount. In one aspect, the therapeutic agent, such as the TAAR agonist, is applied to a skin section, such as a section of the scalp, that contains at least one hair follicle. In a further embodiment, the at least one hair follicle is under tension.

TAAR agonists may be administered to the hair follicle or scalp to promote contraction of the AP muscle and thereby reduce, treat or prevent alopecia and the other disorders discussed herein. It is specifically contemplated that a TAAR agonist or any other agonist of smooth muscle contraction known in the art or disclosed herein can be administered to the hair follicle or the scalp in combination with an agent that retards systemic absorption of the agent across the dermis. In this manner, agents that might otherwise have unwanted systemic effects can be used to treat, reduce or prevent alopecia or other disorders discussed herein while avoiding such systemic side effects. One formulation of agents for topical administration in a manner that avoids systemic absorption is discussed in detail in U.S. 2009/0068287, which is incorporated herein by reference in its entirety.

In another aspect, described herein is a method for prevention of traction alopecia comprising: applying a therapeutically effective amount, such as a pilomotor effective amount, of a TAAR agonist to the scalp to an area with a group of follicles that will experience a pulling force from a hair augmentation device; and attaching the hair augmentation device to the group of follicles. In one embodiment, the hair augmentation device is a hair extension or extensions. In another embodiment the hair augmentation device is a weave. In another embodiment, the hair augmentation device is a barrette.

In another aspect, described herein is method of reducing hair shedding, such as occurs during brushing, combing, weaving, flat ironing, showering, curling, wift, attaching hair extensions or wigs, trading, pony tails, or cosmetic procedures, the method comprising applying a therapeutically effective amount, such as a pilomotor effective amount, of a TAAR agonist topically to a portion of skin that includes at least one hair follicle. The method of reducing shedding can involve reducing shedding at or near the frontal hair line as a result of mechanical forces induced by wearing a ponytail hairstyle. For example, the method can involve treating (reducing or preventing) frontal pattern traction alopecia caused by a ponytail style hair. In one embodiment, the TAAR agonist is present on a brush or comb that may then be used to administer the therapeutic agent such as the TAAR agonist. In another embodiment, the TAAR agonist is applied to the skin prior to the brushing or combing. In another embodiment, the TAAR agonist is applied to the skin without a step of brushing or combing afterwards. Some embodiments can involve applying the TAAR agonist to the skin, which can further involve allowing the TAAR agonist to sit or be absorbed into the skin. Alternatively, the TAAR agonists can be agitated (e.g., massaged, combed, brushed, etc.) into the skin. Applying the TAAR agonist to the skin can involve applying it at or near a hair line of an individual. For example, any of the embodiments of the composition disclosed herein (e.g., lotion, gel, hair product, etc.) can be applied to the skin of an individual at or near the hair line. This can be done for the treatment of alopecia. The alopecia may be caused by mechanical stress induced by ponytail hair styles or other hair styles.

As a non-limiting, example, the TAAR agonist can be formulated as a composition comprising a moisturizing lotion, a facial cream, a sunscreen, a gel, an ointment, a foam, a spray, etc. (which can include a cosmetic formulation) configured to be applied at or near a hair line of an individual (male or female) to treat or prevent frontal pattern traction alopecia (e.g., frontal pattern traction alopecia caused by a ponytail style hair). The composition can be applied to the skin and allowed to sit or be absorbed into the skin, be massaged in the skin, be combed or brushed into the hair, etc.

In another aspect, the cosmetic procedure is selected from the group consisting of brushing, braiding, flat ironing, and combinations of two or more thereof. The therapeutic agent, such as the TAAR agonist, may be topically applied once, twice, or more often per day. In another embodiment, the TAAR agonist is applied to the skin twice daily. In another embodiment, the TAAR agonist is applied to the skin prior to the cosmetic procedure.

In another aspect, described herein is a method for treatment of trichotillomania comprising applying a pilomotor effective amount of a TAAR agonist topically to a portion of skin that includes at least one hair follicle.

The disclosure also concerns evaluating an individual for susceptibility to treatment according to the methods disclosed herein. In one embodiment, the method comprises (1) applying a TAAR agonist (e.g., without limitation, octopamine, tyramine, etc.) on a site on the skin of a person; and (2) 30 to 60 minutes after applying, observe whether the person's skin shows goosebumps or pilioerection at the site; wherein if pilioerection or goosebumps are observed, diagnosing the person as likely to be a successful candidate for use of the trace amine associated receptor agonist for any of the many methods of treatment or prevention described herein. This method may be combined with any of the other methods of treatment or prevention or reduction of hair loss described herein to provide an initial diagnosis of those people most likely to benefit from the methods described. The step of application to the skin may be, in one embodiment, applying a bandage or patch coated with the TAAR agonist to the person's arm or thigh. In another embodiment of any composition or method involving a TAAR agonist, the agonist is octopamine or tryptamine.

During or after the hair experiences stress, the compositions discloses herein may be washed out, such as by shampooing. In some embodiments, the compositions of the present disclosure can be liquid solutions. The liquid solution may be applied directly to the scalp and rubbed into the scalp, or applied by spraying on with a delivery device such as a pump sprayer. The composition of the present disclosure may further be combined with a pre-shampoo tonic, a shampoo, or a conditioner or other hair care product to create a product that has more than one function.

In some embodiments, the compositions described herein are provided as a kit. A “kit” can include a package having at least one composition of the invention and another item useful in its application, such as a comb, brush or other applicator, or with another hair care composition product, such as a shampoo, hair color/dye, hair oil or conditioner. For example, a kit may be a package containing at least one composition of the invention and a spray container or a dropper for administering the composition. In another embodiment, the kit is a package containing (1) the present composition and (2) one or more of a shampoo, hairspray, conditioner, detangling solution, hair color, henna, or hair oil (such as without limitation coconut oil, jojoba oil, olive oil, baby oil, and black castor oil) and optionally (3) a pump spray container holding the present composition or suitable to hold the composition.

### Formulations

The therapeutic agents, particularly the TAAR agonists, described herein and used in the present methods may be formulated into compositions according to the knowledge of one of skill in the art. In one embodiment, the TAAR agonist or other stimulator of AP muscle contraction is formulated for topical slow or prolonged release. As but one example, in one embodiment the AP stimulating agent is encapsulated for slow release and integrated into a hair extension.

In another embodiment, the TAAR agonist or other stimulator of AP muscle contraction is formulated in a shampoo (which can reduce hair shedding during hair brushing), a foam, ointment, spray, solution, gel, slow release capsule, oral tablet, or any similar compound or delivery vehicle or methodology. Topical application is preferred. In one embodiment, the composition is formulated in a topical cream. In another embodiment, the composition is formulated in a hair styling product selected from the group consisting of a styling gel, a styling foam, and a hair conditioner. In another embodiment, the composition is formulated as a hair treating product selected from the group consisting of pre-shampoo tonic, shampoo, and a conditioner.

In another embodiment, the composition may comprise an exfoliating agent to promote abrasion of the surface of the scalp. Examples of the exfoliating agent include (1) inorganic and/or metallic particles such as: boron nitride, in body-centered cubic form (Borazon®); aluminosilicate (e.g. nepheline); zircon; mixed oxides of aluminum such as emery; zinc oxide; aluminum oxides such as aluminas or corundum; titanium oxide; titanium oxide coated mica; carbides, in particular silicon carbide (carborundum); or other metal oxides; metals, and metal alloys such as iron shot, steel shot, and in particular perlite; silicates such as glass, quartz, sand, or vermiculite; calcium carbonate (e.g. Bora-Bora sand or Rose de Brignoles sand) or magnesium carbonate; sodium chloride; pumice stone; amorphous silica; diamond; ceramics, and (2) organic particles such as: fruit stones, in particular apricot stones, e.g. Scrubami® apricot; wood cellulose, e.g. ground bamboo stem; coconut shell, e.g. coconut exfoliator; polyamides, in particular Nylon-6; sugars; plastic microbeads, e.g. polyethylenes or polypropylenes; ground walnut; ground apricot seed; ground shells, and (3) mixed particles associating organic and inorganic compounds, and particles coated in the above compounds. The exfoliating agents may be in the form of microbeads of less than five millimeters in its largest dimension that have an exfoliating effect.

In one embodiment, the composition comprising a TAAR agonist can be formulated as a drug. In one embodiment, the composition comprising a TAAR agonist can be formulated as a cosmetic product.

In one embodiment, the composition comprising a TAAR agonists can be formulated as a pharmaceutical or cosmetic agent, which can include formulating the TAAR agonist with any one or combination of abacavir, acebutolol, acetaminophen, acetaminosalol, acetazolamide, acetohydroxamic acid, acetylsalicylic acid, N-acylglutathione ethyl ester and other esters, N-acyl proline ethyl ester and other esters, acitretin, aclovate, acrivastine, actiq, acyclovir, adalimumab, adapalene, adefovir dipivoxil, adenosine, albuterol, alefacept, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, p-aminobenzoic acid, aminocaproic acid, aminolevulinic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, benzilic acid, bendroflumethiazide, benzocaine, benzonatate, benzophenone, benzoyl peroxide, benztropine, bepridil, betamethasone dipropionate, betamethasone valerate, botulinum toxin, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clocortolone pivalate, clomiphene, clonidine, clopidogrel, clotrimazole, clozapine, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, diclofenac, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, efalizumab, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, etanercept, ethacrynic acid, ethinyl estradiol, ethyl pyruvate, etidocaine, etomidate, famciclovir, famotidine, felodipine, fentanyl, ferulic acid, fexofenadine, finasteride, flecamide, fluconazole, flucytosine, fluocinolone acetonide, fluocinonide, 5-fluorouracil, fluoxetine, fluphenazine, flurazepam, fluticasone propionate, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, infliximab, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loratadine, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mequinol, mercaptopurine, mescaline, metanephrine, metaproterenol, metaraminol, metformin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nitrofurantoin, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate 0, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, phenelzine, pheniramine, phenmetrazine, phenobarbital, phenol, phenoxybenzamine, phentolamine, phenylephrine, phenylpropanolamine, phenyloin, N-(phosphonomethyl)-glycine, N-(phosphonomethyl)-creatine, N-(phosphonomethyl)-tyramine, physostigmine, pilocarpine, pimecrolimus, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocalne, procainamide, procaine, procarbazine, praline, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertaconazole, sertindole, sertraline, shale tar, sibutramine, sildenafil, sotalol, streptomycin, strychnine, sulconazole, sulfacetamide, sulfabenz, sulfabenzamide, sulfabromomethazine, sulfacetamide (sodium sulfacetamide), sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tacrolimus, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, thalidomide, theobromine, theophylline, thiabendazole, thioctic acid (lipoic acid), thioridazine, thiothixene, thymol, tiagabine, timolol, tinidazole, tioconazole, tirofiban, tizanidine, tobramycin, tocamide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan, and zolpidem.

The amount of therapeutic agent present in the composition may be determined by one of skill in the art using known methodologies. In certain embodiments, the TAAR agonist or other stimulator of AP muscle contraction is present in the composition in a concentration from about 0.20% to 0.30%, or about 0.25% by weight. In another embodiment, the therapeutic agent such as a TAAR agonist is present in the composition in a concentration of about 0.25%, 0.33%, 0.5%, 1%, 2%, 2.5%, or 10% by weight.

In other embodiments, the therapeutic agent, such as the TAAR agonist, is present in the topical composition for use in the methods disclosed herein in a concentration from about 0.1% to 35%, about 1.0% to 30%, about 0.2% to 30%, about 0.2% to 25%, about 0.2% to 20%, about 0.2% to 15%, about 0.2% to 10%, about 0.2% to 5%, about 0.2% to 4%, about 0.2% to 3%, about 0.2% to 2%, about 0.2% to 1%, about 10.0% to 30%, about 15.0% to 30%, about 20.0% to 30%, about 10% to 20%, about 10% to 15%, about 15% to 20%, about 15% to 60%, about 20% to 60%, about 50% to 60%, and about 45% to 55% by weight. For certain therapeutic agents, such as octopamine, or tyramine (racemic mixture), a concentration of about 25% to 60%, 30% to 50%, 30% to 60%, 25% to 30%, 40% to 50%, or 50% to 55% by weight of the total weight of the composition is desirable.

In one embodiment, the composition comprises a TAAR agonist in a concentration of about 0.25%, about 0.33%, about 0.5%, about 1%, about 2%, about 2.5%, about 3.0%, about 4.0%, about 10%, about 15%, about 20%, or about 25% by weight.

The compositions used in the present disclosure, particularly compositions containing a TAAR agonist, may be formulated with a preservative such as EDTA (0.1-0.5% by weight of the formulation) and/or sodium metabisulfite (0.1-0.5% by weight of the formulation). In some embodiments, the penetration enhancer is selected from one or more of the group consisting of alcohols, glycols, fatty acids, fatty esters, fatty ethers, occlusive agents, surface active agents, dimethylaminopropionic acid derivatives, terpenes, sulfoxides, cyclic ethers, amides, and amines. Other components of the formulations used herein may be chosen from cosmetically approved excipients known in the art, including water, thickeners, etc.

The composition may be packaged in a kit with an applicator for application to the skin. The invention is also directed to a kit comprising a composition of the therapeutic agent, such as a TAAR agonist, and an applicator, and to a kit comprising a composition of the therapeutic agent, such as a TAAR agonist, and a hair brush or comb, particularly a brush or comb that provides exfoliating effect on the scalp such that there is light abrasion after its use that enhances penetration of the therapeutic agent to the AP muscle. In one embodiment, the therapeutic agent is provided in a metered dose applicator that provides for a fixed volume of the composition to be administered with each administration, such as 1 ml of the topical composition per administration.

It will be understood that the ranges described above, and throughout this document, are also intended to encompass single values contained within these ranges. For example, for a formulation comprising a particular ingredient in a range between 1-50%, a percentage of 5% or 49% is also intended to be disclosed.

### Therapeutic Agents

The methods of the present disclosure may be used with a TAAR agonist or other compound that causes contraction directly or indirectly of the AP muscle. Suitable TAAR agonists can be utilized including citrus aurantium (e.g. bitter orange extract), 2-phenylethylamine, tyramine, p-tyramine, m-tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamylamine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO5166017, RO5203648, RO5256390, RO5263397, RO5212773 (EPPTB), etc. Other suitable TAAR agonists can be found at: Mark D. Berry, et al. Pharmacology of Human Trace Amine-associated Receptors: Therapeutic Opportunities and Challenges. Pharmacology & Therapeutics 18 (2017) 161-180, the entire contents of which is incorporated herein by reference in its entirety. Additionally, derivatives of TAAR agonists can be utilized including derivatives of the compounds mentioned above. In other embodiments, a prodrug that is activated to become a TAAR agonist can be utilized. For example, midodrine is one such prodrug. A particular prodrug can be activated by endogenous enzymes in the scalp such as Caspase-1 when follicular inflammation is present, e.g., at the location of application of a hair extension. In one embodiment, the TAAR agonist is octopamine. In one embodiment, the TAAR is phenyethylamine or octopamine, including compositions comprising the 1-enantiomer of octopamine, that are essentially free of other enantiomers of octopamine, or in which less than 30%, 25%, 20%, 15%, 10%, 12%, 5%, 3%, 1%, or 0.5% by weight of the octopamine present in the composition is a different enantiomer. The octopamine enantiomer R-(−)-4-(2-amino-1-hydroxyethyl)phenol may be obtained from natural bitter orange extract.

In one embodiment, the TAAR agonist is tyramine, or a pharmaceutically acceptable salt or hydrate thereof. Other agents can be 4-(2-Aminoethyl)phenol, 51-67-2, 4-Hydroxyphenethylamine, P-Tyramine, 2-(4-Hydroxyphenyl)ethylamine, Hydroxyphenethylamine, 4-(2-Aminoethyl)phenol, 4-Hydroxyphenethylamine, p-Tyramine, para-Tyramine, Tyramine, 4-(2-Aminoethyl)phenol, 51-67-2, 4-Hydroxyphenethylamine, p-Tyramine, 2-(4-Hydroxyphenyl)ethylamine, Uteramine, Tyramin, Tyrosamine, Tocosine, 4-Hydroxyphenylethylamine, Systogene, Phenol, 4-(2-aminoethyl)-p-Hydroxyphenethylamine, Tenosin-wirkstoff, p-Hydroxyphenylethylamine, p-(2-Aminoethyl)phenol, 2-(p-Hydroxyphenyl)ethylamine, Phenethylamine, p-hydroxy-p-beta-Aminoethylphenol, Phenol, p-(2-aminoethyl)-Benzeneethanamine, 4-hydroxy-Tyramine base, beta-Hydroxyphenylethylamine, NSC 249188, alpha-(4-Hydroxyphenyl)-beta-aminoethane, UNII-X8ZC7V0OX3, [3H]tyramine, [3H]-Tyramine, BRN 1099914, etc.

In one embodiment, the TAAR agonist is octopamine or tyramine, or a pharmaceutically acceptable salt or hydrate thereof, in a composition in a concentration of 0.25% to 40%, 0.25% to 25% by weight, or 0.5% to 22.5% by weight, or 0.75% to 20% by weight, or 1% to 17.5% by weight, or 1.5% to 15% by weight, or 2% to 14.5% by weight, or 2.5% to 14% by weight, or 5% to 13.5% by weight, or 7.5% to 12.5% by weight, or 8% to 12% by weight, or 8.5% to 11.5% by weight, or 9% to 11% by weight, or 9.25% to 10.75% by weight, or 9.5% to 10.5% by weight, or 9.6% to 10.4% by weight, or 9.7% to 10.3% by weight, or 9.8% to 10.2% by weight, or 9.9% to 10.1% by weight, or 9.95% to 10.05% by weight, or 9.96% to 10.04% by weight, or 9.97% to 10.03% by weight, or 9.98% to 10.02% by weight, or 9.99% to 10.01% by weight.

In one embodiment, the TAAR agonist is octopamine or tyramine, or a pharmaceutically acceptable salt or hydrate thereof, in a composition in a concentration at a range of 0.25%, 0.5%, 0.75%, 1%, 1.5%, 2%, 2.5%, 5%, 7.5%, 8%, 8.5%, 9%, 9.25%, 9.5%, 9.6%, 9.7%, 9.8%, 9.9%, 9.95%, 9.96%, 9.97%, 9.98%, or 9.99% by weight as the lower weight limit of the range to an upper weight limit of 10.01%, 10.02%, 10.03%, 10.04%, 10.05%, 10.1%, 10.2%, 10.3%, 10.4%, 10.5%, 10.75%, 11%, 11.5%, 12%, 12.5%, 13.5%, 14%, 14.5%, 15%, 17.5%, 20%, 22.5%, 25%, 30%, 35%, 40%, 45%, or 50% by weight (e.g., a range of 0.25% to 10.01%, 0.25% to 10.02%, 0.5% to 10.01%, 0.5% to 10.02%, etc.).

In one embodiment, the TAAR agonist is octopamine or tyramine, or a pharmaceutically acceptable salt or hydrate thereof, in a composition in a concentration of 0.25% by weight, or 0.5% by weight, or 0.75% by weight, or 1% by weight, or 1.5% by weight, or 2% by weight, or 2.5% by weight, or 5% by weight, or 7.5% by weight, or 8% by weight, or 8.5% by weight, or 9% by weight, or 9.25% by weight, or 9.5% by weight, or 9.6% by weight, or 9.7% by weight, or 9.8% by weight, or 9.9% by weight, or 9.95% by weight, or 9.96% by weight, or 9.97% by weight, or 9.98% by weight, or 9.99% by weight, or 10% by weight, or 10.01% by weight, or 10.02% by weight, or 10.03% by weight, or 10.04% by weight, or 10.05% by weight, or 10.1% by weight, or 10.2% by weight, or 10.3% by weight, or 10.4% by weight, or 10.5% by weight, or 10.75% by weight, or 11% by weight, or 11.5% by weight, or 12% by weight, or 12.5% by weight, or 13.5% by weight, or 14% by weight, or 14.5% by weight, or 15% by weight, or 17.5% by weight, or 20% by weight, or 22.5% by weight, or 25% by weight, or 30% by weight, or 40% by weight, or 45% by weight, or 50% by weight, or 55% by weight.

In another embodiment, the composition comprises a TAAR agonist that is octopamine or tyramine, or a pharmaceutically acceptable salt or hydrate thereof, or that comprises one enantiomer of octopamine or tyramine, namely R-(−)-4-(2-amino-1-hydroxyethyl)phenol and is substantially free of other enantiomer(s) of octopamine or has less than 30%, 25%, 20%, 15%, 10%, 12%, 5%, 3%, 1%, or 0.5% by weight of the octopamine or tyramine present in the composition as a different enantiomer, wherein the octopamine or tyramine is present in the composition in a concentration of 30% to 70% by weight, or 35% to 65% by weight, or 37.5% to 62.5% by weight, or 40% to 60% by weight, or 42.5% to 57.5% by weight, or 45% to 55% by weight, or 45.5% to 54.5% by weight, or 46% to 54% by weight, or 46.5% to 53.5% by weight, or 47% to 53% by weight, or 47.5% to 52.5% by weight, or 48% to 52% by weight, or 48.25% to 51.75% by weight, or 48.5% to 51.5% by weight, or 48.75% to 51.25% by weight, or 49% to 51% by weight, or 49.25% to 50.75% by weight, or 49.5% to 50.5% by weight, or 49.6% to 50.4% by weight, or 49.7% to 50.3% by weight, or 49.8% to 50.2% by weight, or 49.9% to 50.1% by weight.

In another embodiment, the composition comprises a TAAR agonist that is octopamine or tyramine, or a pharmaceutically acceptable salt or hydrate thereof, or that comprises one enantiomer of octopamine or tyramine, namely R-(−)-4-(2-amino-1-hydroxyethyl)phenol and is substantially free of other enantiomer(s) of octopamine or tyramine or has less than 30%, 25%, 20%, 15%, 10%, 12%, 5%, 3%, 1%, or 0.5% by weight of the octopamine present in the composition as a different enantiomer, wherein the octopamine or tyramine is present in the composition in a concentration of 20% by weight, or 25% by weight, or 30% by weight, or 35% by weight, or 37.5% by weight, or 40% by weight, or 42.5% by weight, or 45% by weight, or 45.5% by weight, or 46% by weight, or 46.5% by weight, or 47% by weight, or 47.5% by weight, or 48% by weight, or 48.25% by weight, or 48.5% by weight, or 48.75% by weight, or 49% by weight, or 49.25% by weight, or 49.5% by weight, or 49.6% by weight, or 49.7% by weight, or 49.8% by weight, or 49.9% by weight to 50.1% by weight, or 50.2% by weight, or 50.3% by weight, or 50.4% by weight, or 50.5% by weight, or 50.75% by weight, or 51% by weight, or 51.25% by weight, or 51.5% by weight, or 51.75% by weight, or 52% by weight, or 52.5% by weight, or 53% by weight, or 53.5% by weight, or 54% by weight, or 54.5% by weight, or 55% by weight, or 57.5% by weight, or 60% by weight, or 62.5% by weight, or 65% by weight, or 70% by weight.

In one embodiment, the composition comprises a TAAR agonist that is R-(−)-4-(2-amino-1-hydroxyethyl)phenol substantially free of the other enantiomer of octopamine or tyramine (or having less than 25%, 20%, 15%, 10%, 5%, 1% or 0.1% of the other enantiomer of octopamine or tyramine) or a pharmaceutically acceptable salt or hydrate thereof, in a composition in a concentration of 20% by weight, or 21% by weight, or 25% by weight, or 26% by weight, or 30% by weight, or 35% by weight, or 37.5% by weight, or 40% by weight, or 42.5% by weight, or 45% by weight, or 45.5% by weight, or 46% by weight, or 46.5% by weight, or 47% by weight, or 47.5% by weight, or 48% by weight, or 48.25% by weight, or 48.5% by weight, or 48.75% by weight, or 49% by weight, or 49.25% by weight, or 49.5% by weight, or 49.6% by weight, or 49.7% by weight, or 49.8% by weight, or 49.9% by weight, or 50% by weight, or 50.1% by weight, or 50.2% by weight, or 50.3% by weight, or 50.4% by weight, or 50.5% by weight, or 50.75% by weight, or 51% by weight, or 51.25% by weight, or 51.5% by weight, or 51.75% by weight, or 52% by weight, or 52.5% by weight, or 53% by weight, or 53.5% by weight, or 54% by weight, or 54.5% by weight, or 55% by weight, or 57.5% by weight, or 60% by weight, or 62.5% by weight, or 65% by weight, or 70% by weight.

In another embodiment, the composition comprises a TAAR agonist that is octopamine or tyramine, or a pharmaceutically acceptable salt or hydrate thereof, or that comprises one enantiomer of octopamine or tyramine, namely R-(−)-4-(2-amino-1-hydroxyethyl)phenol and is substantially free of other enantiomer(s) of octopamine or tyramine or has less than 30%, 25%, 20%, 15%, 10%, 12%, 5%, 3%, 1%, or 0.5% by weight of the octopamine present in the composition as a different enantiomer, wherein the octopamine or tyramine is present in the composition in a concentration of 10% to 60% by weight, or 12.5% to 50% by weight, or 10% to 50% by weight, or 15% to 40% by weight, or 20% to 30% by weight, or 20% to 40% by weight, or 17.5% to 30% by weight, or 20% to 25% by weight, or 20.5% to 24.5% by weight, or 21% to 24% by weight, or 21.5% to 23.5% by weight, or 21.75% to 23.25% by weight, or 22% to 23% by weight, or 22.1% to 22.9% by weight, or 22.2% to 22.8% by weight, or 22.3% to 22.7% by weight, or 22.4% to 22.6% by weight.

In another embodiment, the composition comprises a TAAR agonist that is octopamine or tyramine, or a pharmaceutically acceptable salt or hydrate thereof, or that comprises one enantiomer of octopamine or tyramine, namely R-(−)-4-(2-amino-1-hydroxyethyl)phenol and is substantially free of other enantiomer(s) of octopamine or tyramine or has less than 30%, 25%, 20%, 15%, 10%, 12%, 5%, 3%, 1%, or 0.5% by weight of the octopamine present in the composition as a different enantiomer, wherein the octopamine or tyramine is present in the composition in a concentration of 10% by weight, or 12.5% by weight, or 15% by weight, or 17.5% by weight, or 20% by weight, or 20.5% by weight, or 21% by weight, or 21.5% by weight, or 21.75% by weight, or 22% by weight, or 22.1% by weight, or 22.2% by weight, or 22.3% by weight, or 22.4% by weight to 22.6% by weight, or 22.7% by weight, or 22.8% by weight, or 22.9% by weight, or 23% by weight, or 23.25% by weight, or 23.5% by weight, or 24% by weight, or 24.5% by weight, or 25% by weight, or 30% by weight, or 40% by weight, or 50% by weight, or 60% by weight.

In one embodiment, the composition comprises one enantiomer of octopamine, namely R-(−)-4-(2-amino-1-hydroxyethyl)phenol, and is substantially free of other enantiomer(s) of octopamine or tyramine or has less than 30%, 25%, 20%, 15%, 10%, 12%, 5%, 3%, 1%, or 0.5% by weight of the octopamine or tyramine present in the composition as a different enantiomer, wherein the R-(−)-4-(2-amino-1-hydroxyethyl)phenol is present in the composition in a concentration of 20% to 25% by weight.

In a further embodiment, the TAAR agonist is phenylethylamine, or a pharmaceutically acceptable salt or hydrate thereof, in a composition in a concentration of 0.01% to 2% by weight, or 0.02% to 1.75% by weight, or 0.03% to 1.5% by weight, or 0.04% to 1.25% by weight, or 0.05% to 1% by weight, or 0.1% to 0.9% by weight, or 0.15% to 0.85% by weight, or 0.2% to 0.8% by weight, or 0.25% to 0.75% by weight, or 0.3% to 0.7% by weight, or 0.35% to 0.65% by weight, or 0.4% to 0.6% by weight, or 0.41% to 0.59% by weight, or 0.42% to 0.58% by weight, or 0.43% to 0.57% by weight, or 0.44% to 0.56% by weight, or 0.45% to 0.55% by weight, or 0.46% to 0.54% by weight, or 0.47% to 0.53% by weight, or 0.48% to 0.52% by weight, or 0.49% to 0.51% by weight.

In a further embodiment, the TAAR agonist is phenylethylamine, or a pharmaceutically acceptable salt or hydrate thereof, in a composition in a concentration of 0.01% by weight, or 0.02% by weight, or 0.03% by weight, or 0.04% by weight, or 0.05% by weight, or 0.1% by weight, or 0.15% by weight, or 0.2% by weight, or 0.25% by weight, or 0.3% by weight, or 0.35% by weight, or 0.4% by weight, or 0.41% by weight, or 0.42% by weight, or 0.43% by weight, or 0.44% by weight, or 0.45% by weight, or 0.46% by weight, or 0.47% by weight, or 0.48% by weight, or 0.49% by weight to 0.51% by weight, or 0.52% by weight, or 0.53% by weight, or 0.54% by weight, or 0.55% by weight, or 0.56% by weight, or 0.57% by weight, or 0.58% by weight, or 0.59% by weight, or 0.6% by weight, or 0.65% by weight, or 0.7% by weight, or 0.75% by weight, or 0.8% by weight, or 0.85% by weight, or 0.9% by weight, or 1% by weight, or 1.25% by weight, or 1.5% by weight, or 1.75% by weight, or 2% by weight.

In a further embodiment, the TAAR is phenylethylamine, or a pharmaceutically acceptable salt or hydrate thereof, in a composition in a concentration of 0.01% by weight, or 0.02% by weight, or 0.03% by weight, or 0.04% by weight, or 0.05% by weight, or 0.1% by weight, or 0.15% by weight, or 0.2% by weight, or 0.25% by weight, or 0.3% by weight, or 0.35% by weight, or 0.4% by weight, or 0.41% by weight, or 0.42% by weight, or 0.43% by weight, or 0.44% by weight, or 0.45% by weight, or 0.46% by weight, or 0.47% by weight, or 0.48% by weight, or 0.49% by weight, or 0.5% by weight, or 0.51% by weight, or 0.52% by weight, or 0.53% by weight, or 0.54% by weight, or 0.55% by weight, or 0.56% by weight, or 0.57% by weight, or 0.58% by weight, or 0.59% by weight, or 0.6% by weight, or 0.65% by weight, or 0.7% by weight, or 0.75% by weight, or 0.8% by weight, or 0.85% by weight, or 0.9% by weight, or 1% by weight, or 1.25% by weight, or 1.5% by weight, or 1.75% by weight, or 2% by weight.

In some embodiments, provided herein is a TAAR agonist formulated with a carrier or delivery vehicle optimized for delivery of the TAAR agonist to the scalp. A TAAR agonist can be released using several different formulations or release methods including time release, creams, ointments, sprays, capsules, or other release methods. For instance, the TAAR agonist can be incorporated into a shampoo for utilization during showering so that when a user brushes their hair, their follicles will be tightly held by the AP muscles to prevent brushing from unnecessarily pulling out healthy hair. In other embodiments, the TAAR agonist can be included in ointments or other topical creams that could be applied to the scalp so that it can be slowly absorbed into the skin and stimulate the smooth muscle. In other embodiments, the TAAR agonist can be included in a liquid spray or aerosol medium to be applied to the scalp. In other embodiments, the TAAR agonist can be incorporated into capsules or other slow release vehicles that would allow the chemical or agent to be slowly released into the dermis of the scalp. Capsules or vehicles that encapsulate the TAAR agonist can include, but are not limited to, liposomes, non-ionic liposomes, niosomes, novasome I, erythromycin-Zn complex, microspheres, nanoparticles, solid lipid nanoparticles, and nanoemulsions. In some embodiments, this can include a gel or foam that is applied to the scalp. It is specifically contemplated that the TAAR agonist can be formulated in hair care products such as styling gel, styling foam, hair conditioner, hair serum, a hair mask, a hair shampoo, a hair pre-shampoo tonic, etc.

Any of the aforementioned TAAR agonist can be applied by a user before the application of a hair extension device or other device or condition that exerts force on the hair follicle. Alternatively, a TAAR agonist can be used routinely (e.g. twice daily) after such a device has been installed. Routine use of a TAAR agonist would be indicated as a prophylactic against traction alopecia for users of a hair extension device or other device that exerts force on the hair follicle.

Creams or other formulations with different TAAR agonists can be applied prior to a user utilizing a hair piece or brushing the hair. In some embodiments, a hair piece or hair extensions can contain pads or other absorbent material that can absorb TAAR agonist in a foam or cream applied prior to application to a user's head. In other embodiments, slow release capsules can be incorporated into the hair extensions or hair pieces, or can be included in barrettes. In some embodiments, barrettes will include pads with an absorbent layer for application of TAAR agonist cream or other TAAR agonist topical formulation.

Efficacy of treatment to treat or prevent traction alopecia can be determined by monitoring the density of hairs on a given area of the subject's body, e.g., a given area of the scalp. If the rate of hair loss is reduced, e.g., by 10% or more following treatment, the treatment is effective for the prevention of traction alopecia. Similarly, if hair density remains the same, despite ongoing traction that would normally have been expected to cause traction alopecia, the treatment is effective for the prevention of traction alopecia. If the density of hair increases, e.g., by 5% or more, e.g., by 10% or more following treatment and despite ongoing traction, the treatment is also considered effective for the treatment and/or prevention of traction alopecia.

As noted above, it is contemplated that all forms of alopecia can benefit from the technology described herein. For example, the technology described herein can be applicable to prevent or treat androgenic alopecia. The AP muscle degenerates in the process of androgenic alopecia (reviewed, e.g., in Torkamani et al., Int. J. Trichology 6:88-94 (2014)); without wishing to be bound by theory, it is contemplated that regular stimulation of AP muscle contraction may slow or reduce the loss of the muscle and thereby benefit the treatment or prevention of androgenic alopecia.

It is also contemplated that the technology described herein can be broadly applicable to any type of condition of which at least one hair follicle is under tension. Using the TAAR agonist compositions or other agents that stimulate AP muscle contraction as described herein, it is contemplated that one can limit or reduce hair shedding under such conditions.

In one aspect, the condition of which at least one hair follicle is under tension is brushing or combing. Accordingly, the technology described herein relates to a method of reducing hair shedding during brushing or combing. As used herein, the term “reducing hair shedding” means that the amount of hair shedding from a subject is reduced by at least 5%, at least 10%, at least 15%, at least 20%, at least 25%, at least 30%, at least 35%, at least 40%, at least 45%, at least 50%, or more, as compared to what would be expected in the absence of the method. A TAAR agonist or other agent that stimulates AP muscle contraction can be present on the brush or comb used for the brushing or combing. In one embodiment, the TAAR agonist or other agent can be applied to the brush or comb prior to brushing or combing, e.g., in the form of a liquid, gel, cream or spray. In one embodiment, the brush or comb can dispense the TAARA or other agent.

Agents that promote the contraction of the AP muscle can optionally be administered by iontophoresis, which uses an electric field to drive the passage of ionic agents or drugs into the skin. As but one example, iontophoresis has been used to deliver agents such as phenylephrine to the skin to stimulate AP muscle contraction (See, e.g., Siepmann et al., Neurology Apr. 25, 2012; 78(Meeting Abstracts 1): P05.197). Thus, in one embodiment, a brush or comb can incorporate an iontophoresis device, which can dispense the TAARA or other agent and/or be used for transdermal delivery of the agent(s). The iontophoresis device can comprise one or more metal contacts. Optionally, the iontophoresis device can comprise one or more compartments for containing the TAAR agonist or other agent(s).

In another aspect, the condition in which at least one hair follicle is under tension is a hair-related cosmetic procedure. Accordingly, the technology described herein relates to a method of reducing hair shedding during a hair-related cosmetic procedure. Examples of hair-related cosmetic procedures include, but are not limited to, brushing, braiding, flat ironing, and combinations thereof.

In another aspect, the condition in which at least one hair follicle is under tension is trichotillomania, a disorder characterized by the compulsive urge to pull out one's hair. Accordingly, to the extent that increasing the force required to remove the hair can help counter hair loss due to this condition, the stimulation of AP muscle contraction as described herein can provide a method to reduce the hair loss.

### Other Agents or Approaches to Contract the Smooth Muscle

Other agents or approaches can be used to contract the smooth muscle for the prevention or treatment of alopecia, e.g., hair shedding. As noted above, any agent or treatment that stimulates AP muscle contraction is of potential use in methods of treating, reducing or preventing alopecia as described herein.

In one embodiment, the smooth muscle can be contracted by stimulating or activating a cold receptor. A cold receptor can be stimulated, for example, by activating the TRPM8 channel. Exemplary agents that can stimulate a cold receptor include, but are not limited to, menthol and icilin. Compositions and methods for stimulating a cold receptor are disclosed, for example, in U.S. Pat. No. 4,034,109, the contents of which are incorporated by reference in its entirety.

Where the AP muscle is served by or associated with both noradrenergic fibers and a cholinergic system, agents that stimulate release of transmitters from these systems can be used to stimulate AP muscle contraction. Thus, not only TAAR agonists, but also cholinergic agonists, including, but not limited to acetylcholine and other neurotransmitters that stimulate smooth muscle contraction are contemplated for use in the methods and compositions described herein.

The alpha 1 adrenergic receptor is a G protein-coupled receptor. Agonists of other G protein-coupled receptors (e.g., alpha 2 adrenergic receptor) can also be used to stimulate contraction of the smooth muscle. Examples of alpha 2 adrenergic receptor agonists include, but are not limited to, 4-NEMD, 7-Me-marsanidine, agmatine, apraclonidine, brimonidine, clonidine, detomidine, dexmedetomidine, fadolmidine, guanabenz, guanfacine, lofexidine, marsanidine, medetomidine, methamphetamine, mivazerol, rilmenidine, romifidine, talipexole, tizanidine, tolonidine, xylazine, and xylometazoline. As noted above, to the extent that it would be disadvantageous to administer these or other agents systemically, they can be administered in a formulation that permits uptake by the AP muscle in the dermis but limits systemic uptake.

In one embodiment of the present invention a TAAR agonist may be combined with a topically applied alpha adrenergic receptor agonist. Suitable alpha agonists can include cirazoline, desvenlafaxine, etilfrine, metaraminol, methoxamine, naphazoline, oxymetazoline, pseudoephrine, m-synephrine, p-synephrine, synephrine, octopamine, hordenine, tetrahydrozoline, isometheptene, metaraminol, nicergoline, ergonovine, levonordefrin, phendimetrazine, methoxamine, midodrine, clonidine, pergolide, xylometazoline, droxidopa, epinephrine, mephentermine, 4-methoxyamphetamine, Benzphetamine, Naphazoline, Apraclondine, Bromocriptine, Oxymetazoline, Phenylpropanolamine, Pseudoephedrine, Dipivefrin, xylometazoline, etc.

In one embodiment of the present invention a TAAR agonist may be combined with a topically applied alpha adrenergic receptor antagonist. Suitable alpha antagonists can include (+)Dobutamine, abanoquil, Acebutolol, adimolol, ajmalicine, alfuzosin, anisodamine, Atenolol, benoxathian, Betaxolol, Bretylium, Buflomedil, Butoxamine, Carteolol, carvedilol, cirazoline, corynanthine, dihydroergocornine, dihydroergocristine, dihydroergocryptine, dihydroergotoxine, doxazosin, ergot derivatives, Esmolol, Guanadrel, Guanethidine, hydroxymaprotiline, ifenprodil, indoramin, ketanserin, labetalol, Levobunolol, Metoprolol, monatepil, Moxisylyte, Nadolol, nantenine, Nicergoline, oxaprotiline, pelanserin, Penbutolol, phendioxan, phenoxybenzamine, phentolamine, Pindolol, prazosin, Propanolol, pukateine, Raubasine, rauwolscine, Reserpine, silodosin, tamsulosin, terazosin, thiamenidine, tiamenidine, Timolol, Tolazoline, umespirone, urapidil, urapidil, WB-4101, yohimbine, ziprasidone, zuclopenthixol, L-765,314, Z-350, SR 59230A, BMY-7,378.

In one embodiment of the present invention a TAAR agonist may be combined with a topically applied beta adrenergic receptor agonist.

In one embodiment of the present invention a TAAR agonist may be combined with a topically applied beta adrenergic receptor antagonist.

In one embodiment, halostachine (also known as N-methylphenylethanolamine) is contemplated for use as a therapeutic agent in the methods and compositions described herein to stimulate smooth muscle contraction.

It should be noted that agonists described herein also encompass their inorganic or organic salts. Representative salts include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

It should be noted that combinations of the above methods and agents can be used to promote the contraction of the smooth muscle.

It should be noted that combinations of the above methods with an applied TAAR agonist and agents can be used to promote the contraction of the smooth muscle.

### Treatment of Acne

The compositions described herein can also be used for the treatment of acne. It is known that contraction of the AP muscle plays a role in the secretion of the sebum (see Mahfouz et al., J. Egypt wom. Dermatol. Soc. 2005, 2, 25-29). For example, the AP muscle, when contracted, can push on the sebaceous lobule between the hair follicle and the AP muscle, which can exact pressure on the sebaceous lobule. The compositions can be applied in the form of lotion, cream, spray, or wipe. The compositions can be used in combination with benzoyl peroxide or other topical medications for acne treatment.

### Generating Trace Amine Via Local Flora

It is contemplated for some embodiments to include the use of an agent that can induce local flora to generate trace amine. For example, an agent such as a probiotic, a genetically modified (GMO) bacteria, or a viral vector can be used to induce local flora in a person in need of treatment so as to cause generation of a TAAR. Using an agent to induce the generation of TAAR can be done instead of applying a topical composition containing a TAAR agonist or in addition to applying the topical composition containing the TAAR agonist.

In some embodiments, methods disclosed herein for identifying a candidate TAAR (e.g., one that causes goosebumps in a subject) and a candidate patient (e.g., one that experiences goosebumps) can be used to select the TAAR for a specific treatment. A probiotic, a GMO bacteria, and/or a viral vector can then be selected based on its ability to cause local flora in that candidate patient to generate the trace amine associated with that TAAR.

In some embodiments, the composition is formulated to include tyramine as a TAAR agonist. The formulation can include 15% tyramine HCl solution. The composition having 15% tyramine HCl solution can be formulated as a shampoo or a pre-shampoo tonic, for example. One exemplary embodiment of the composition includes 69.25% water, 15% tyramine HCl, 0.10% tetrasodium EDTA, 0.30% sodium metabisulfite, 10.0% PEG-6 caprylic/capric glyceride, 0.25 fragrance, 1.00% polysorbate 80, 0.1% butylated hydroxytolulene, 3.00% 1,3 propanediol, and 1.00% phenoxyethanol ethylhexylglycerin.

In some embodiments, the composition is formulated to include tyramine as a TAAR agonist. The formulation can include 20% tyramine HCl solution. The composition having 20% tyramine HCl solution can be formulated as a skin lotion, gel, or shampoo, for example.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that can have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

### EXAMPLES

### Example 1: p-Octopamine HCl at 5%, 10%, 20% by Weight

A study was conducted to assess the dosage of topical p-octopamine solution required to elicit the pilomotor reflex of the hair arrecto-pili muscle. Five subjects participated in the study. Three formulations were used: Formula A: 5% topical p-octopamine hydrochloride solution; Formula B: 10% topical p-octopamine hydrochloride solution; Formula C: 20% topical p-octopamine hydrochloride solution.

The study was conducted over 3 days. On day 1, subjects were instructed to apply Formula A to their arm. On day 2, subjects were instructed to apply Formula B to their arm. On day 3, subjects were instructed to apply Formula C to their arm. 0.1 mL of each formula was applied using a metered dosage dispenser to each arm. Table 1 summarizes the finding from this study.

TABLE 1
p-octopamine study

| Subject No. | Formula A | Formula B | Formula C |
|---|---|---|---|
| 1 | NR | NR | R |
| 2 | NR | NR | R |
| 3 | NR | NR | R |
| 4 | NR | NR | R |
| 5 | NR | NR | R |

R = Response, i.e. goose bumps; NR = No response

The 20% topical p-octopamine solution (Formula C) elicited a clinical response in all subjects while the 5% and 10% formulations (Formula A and B) failed to elicit a response. With the 20% topical oxymetazoline solution, response in the contraction of the arrector-pilomotor muscle was obtained approximately within 10 minutes and lasted over 4 hours.

Due to the long acting effect of p-octopamine it may be beneficial to apply once daily, every other day, or as needed prior to mechanical procedures that may exert epilatory forces on hair follicles.

### Example 2: m-Octopamine HCl at 1%, 5%, 10% by Weight

A study was conducted to assess the dosage of topical m-octopamine solution required to elicit the pilomotor reflex of the hair arrecto-pili muscle. Five subjects participated in the study. Three formulations were used: Formula A: 1.0% topical m-octopamine hydrochloride solution; Formula B: 5% topical m-octopamine hydrochloride solution; Formula C: 10.0% topical m-octopamine hydrochloride solution.

The study was conducted over 3 days. On day 1, subjects were instructed to apply Formula A to their arm. On day 2, subjects were instructed to apply Formula B to their arm. On day 3, subjects were instructed to apply Formula C to their arm. 0.1 mL of each formula was applied using a metered dosage dispenser to each arm. Table 2 summarizes the finding from this study.

TABLE 2
m-octopamine study—1

| Subject No. | Formula A | Formula B | Formula C |
|---|---|---|---|
| 1 | NR | NR | R |
| 2 | NR | NR | R |
| 3 | NR | NR | R |
| 4 | NR | NR | R |
| 5 | NR | NR | R |

R = Response, i.e. goose bumps; NR = No response

The 10.0% topical m-octopamine solution (Formula C) elicited a clinical response in all subjects while the 1.0% and 5% formulations (Formula A and B) failed to elicit a response. With the 10.0% topical m-octopamine solution, response in the contraction of the arrector-pilomotor muscle was obtained approximately within 5-10 minutes and lasted over 4 hours.

### Example 3: m-Octopamine HCl at 10% by Weight with Antagonist (Alfuzosin) at 0.1%, 0.2%, 0.5%

A study was conducted to assess the dosage of topical m-octopamine solution required to elicit the pilomotor reflex in the presence of an alpha adrenergic receptor antagonist (Alfuzosin) of the hair arrecto-pili muscle. Five subjects participated in the study. Three formulations were used: Formula A: 10% topical m-octopamine hydrochloride and 0.1% Alfuzosin solution; Formula B: 10% topical m-octopamine hydrochloride and 0.2% Alfuzosin solution; Formula C: 10.0% topical m-octopamine hydrochloride and 0.5% Alfuzosin solution.

The study was conducted over 3 days. On day 1, subjects were instructed to apply Formula A to their arm. On day 2, subjects were instructed to apply Formula B to their arm. On day 3, subjects were instructed to apply Formula C to their arm. 0.1 mL of each formula was applied using a metered dosage dispenser to each arm. Table 3 summarizes the finding from this study.

TABLE 3
m-octopamine with alpha antagonist study—1

| Subject No. | Formula A | Formula B | Formula C |
|---|---|---|---|
| 1 | R | R | R |
| 2 | R | R | R |
| 3 | R | R | R |
| 4 | R | R | R |
| 5 | R | R | R |

R = Response, i.e. goose bumps; NR = No response

All 10.0% topical m-octopamine solutions (Formulas A, B, and C) elicited a clinical response in all subjects with 0.1%, 0.2% and 0.5% of an alpha aderenergic antagonist (Alfuzosin) present. With the 10.0% topical m-octopamine with alpha antagonist solution, response in the contraction of the arrector-pilomotor muscle was obtained approximately within 5-10 minutes and lasted over 4 hours.

### Example 4: p-Octopamine HCl at 20% by Weight with Antagonist (Alfuzosin) at 0.1%, 0.2%, 0.5%

A study was conducted to assess the dosage of topical p-octopamine solution required to elicit the pilomotor reflex in the presence of an alpha adrenergic receptor antagonist (Alfuzosin) of the hair arrecto-pili muscle. Five subjects participated in the study. Three formulations were used: Formula A: 20% topical p-octopamine hydrochloride and 0.1% Alfuzosin solution; Formula B: 20% topical p-octopamine hydrochloride and 0.2% Alfuzosin solution; Formula C: 20.0% topical p-octopamine hydrochloride and 0.5% Alfuzosin solution.

The study was conducted over 3 days. On day 1, subjects were instructed to apply Formula A to their arm. On day 2, subjects were instructed to apply Formula B to their arm. On day 3, subjects were instructed to apply Formula C to their arm. 0.1 mL of each formula was applied using a metered dosage dispenser to each arm. Table 4 summarizes the finding from this study.

TABLE 4
p-octopamine with alpha antagonist study—1

| Subject No. | Formula A | Formula B | Formula C |
|---|---|---|---|
| 1 | R | R | R |
| 2 | R | R | R |
| 3 | R | R | R |
| 4 | R | R | R |
| 5 | R | R | R |

R = Response, i.e. goose bumps; NR = No response

All 20.0% topical p-octopamine solutions (Formulas A, B, and C) elicited a clinical response in all subjects with 0.1%, 0.2% and 0.5% of an alpha aderenergic antagonist (Alfuzosin) present. With the 20.0% topical p-octopamine with alpha antagonist solution, response in the contraction of the arrector-pilomotor muscle was obtained approximately within 5-10 minutes and lasted over 4 hours.

### Example 5: m-Octopamine HCl at 10% by Weight

In another study, 10.0% m-octopamine hydrochloride was used to assess the use of topical m-octopamine hydrochloride solution as a novel agent for prevention/reduction of hair loss from mechanical pulling. Participants included in the study were female subjects between ages of 18 and 60 who frequently use traumatic hair care practices, such as tight braids, head scarves, ponytails, extensions, hair rollers, hair weaves and heated styling appliances such as blow dryers, flat irons, heat setters and curling irons. Excluded subjects were those who experienced uncontrolled hypertension, those who were pregnant or breastfeeding, those who were diagnosed with pattern hair loss, or those who experienced other hair loss in conjunction with female pattern hair loss. Overall, fifteen female subjects, aged 24 to 40 years, participated in the study.

Referring to FIGS. 2-5, the study was conducted over 4 days. On day 1, subjects were instructed to wash their hair. On day 2, subjects were instructed to apply 1 mL of placebo solution containing vehicle and brush targeted area after 30 minutes. Brushing was conducted to frontal hair with regular brush in size of 8×10 cm². On day 3, subjects were instructed again to wash their hair. On day 4, subjects were instructed to apply 1 mL of 10% m-octopamine hydrochloride solution on targeted area and brush after 30 minutes. It should be noted that FIGS. 4-5 demonstrate an increase in epilatory force threshold of 172% with a topical application of phenylephrine (PE) as the TAAR agonists. As will be demonstrated with the examples disclosed herein, other TAAR agonists can be used to generate increases in the epilatory force threshold.

FIGS. 2 and 3 show that application of the 10% m-octopamine hydrochloride solution resulted in reduced hair shedding in 80% of the patients, as compared to the placebo solution containing the vehicle, with the average reduction being approximately 42%. Again, FIGS. 4 and 5 show that the epliatory force threshold for plucking hair follicles following topical PE application increased by 172%. The study with the 10% m-octopamine hydrochloride application increased the epilatory force threshold by approximately 200%. Therefore, there is a significant reduction hair loss from mechanical pulling and increase in epilatory force after topical application of 10% m-octopamine hydrochloride. This novel study demonstrates the utility of TAAR agonists in the treatment of traction alopecia and excessive hair loss resulting from mechanical cosmetic procedures.

### Example 6: p-Octopamine HCl at 20% by Weight

Again, referring to FIGS. 2-5, in another study, 20.0% p-octopamine hydrochloride to assess the use of topical p-octopamine hydrochloride solution as a novel agent for prevention/reduction of hair loss from mechanical pulling. Participants included in the study were female subjects between ages of 18 and 60 who frequently use traumatic hair care practices, such as tight braids, head scarves, ponytails, extensions, hair rollers, hair weaves and heated styling appliances such as blow dryers, flat irons, heat setters and curling irons. Excluded subjects were those who experienced uncontrolled hypertension, those who were pregnant or breastfeeding, those who were diagnosed with pattern hair loss, or those who experienced other hair loss in conjunction with female pattern hair loss. Overall, fifteen female subjects, aged 24 to 40 years, participated in the study.

The study was conducted over 4 days. On day 1, subjects were instructed to wash their hair. On day 2, subjects were instructed to apply 1 mL of placebo solution containing vehicle and brush targeted area after 30 minutes. Brushing was conducted to frontal hair with regular brush in size of 8×10 cm². On day 3, subjects were instructed again to wash their hair. On day 4, subjects were instructed to apply 1 mL of 20% p-octopamine hydrochloride solution on targeted area and brush after 30 minutes.

The application of the 20% p-octopamine hydrochloride solution resulted in reduced hair shedding in 80% of the patients, as compared to the placebo solution containing a vehicle only, with the average reduction being approximately 42%. The epliatory force threshold for plucking hair follicles following topical 20% p-octopamine hydrochloride application increased by approximately 240%. Therefore, there is a significant reduction hair loss from mechanical pulling and increase in epilatory force after topical application of 20% p-octopamine hydrochloride. This novel study demonstrates the utility of TAAR agonists in the treatment of traction alopecia and excessive hair loss resulting from mechanical cosmetic procedures.

### Example 7: Phenylephrine and p-Octopamine

Female subjects, ages 18-40, were recruited to study the effect of topically applied p-octopamine and phenylephrine, a selective al-AR agonist, on epilation force and hair shedding during cosmetic procedures. In the blinded study, 80% of subjects demonstrated reduced shedding on days using phenylephrine compared to days using a placebo solution. The average reduction in hair loss was approximately 42%. In addition, the force threshold required for epilation increased by approximately 172% following topical p-octopamine and phenylephrine application. This study demonstrating the utility of al-AR agonists in combination with TAAR agonist in the treatment of traction alopecia and hair shedding during cosmetic procedures.

### Example 8: Tyramine HCl at 5%, 10%, 12% by Weight

A study was conducted to assess the dosage of topical tyramine HCL solution required to elicit the pilomotor reflex of the hair arrecto-pili muscle. Five subjects participated in the study. Three formulations were used: Formula A: 5% topical tyramine hydrochloride solution; Formula B: 10% topical tyramine hydrochloride solution; Formula C: 12% topical tyramine hydrochloride solution. The remaining solution was ethanol.

The study was conducted over 3 days. On day 1, subjects were instructed to apply Formula A to their arm. On day 2, subjects were instructed to apply Formula B to their arm. On day 3, subjects were instructed to apply Formula C to their arm. 0.1 mL of each formula was applied using a metered dosage dispenser to each arm. Table 5 summarizes the finding from this study.

TABLE 5
Tyramine study

| Subject No. | Formula A | Formula B | Formula C |
|---|---|---|---|
| 1 | NR | R | R |
| 2 | NR | NR | R |
| 3 | NR | R | R |
| 4 | NR | R | R |
| 5 | NR | R | R |

R = Response, i.e. goose bumps; NR = No response

The 10% and 12% topical tyramine solution (Formula B and C) elicited a clinical response in all subjects while the 5% (Formula A) failed to elicit a response. With the 12% topical tyramine solution, response in the contraction of the arrector-pilomotor muscle was obtained approximately within 20 minutes and lasted over 4 hours.

### Example 9: Tyramine HCL 12% Solution

Female subjects, ages 18-40, were recruited to study the effect of topically applied tyramine, a selective TAAR agonist, on epilation force and hair shedding during cosmetic procedures. In the blinded study, 80% of subjects demonstrated reduced shedding on days using tyramine compared to days using a placebo solution. The average reduction in hair loss was approximately 38%. In addition, the force threshold required for epilation increased by approximately 376% following topical tyramine. This study demonstrating the utility of TAAR agonists in the treatment of traction alopecia and hair shedding during cosmetic procedures.

While specific examples of certain TAAR agonists and other agents are disclosed in the examples, one skilled in the art will appreciate that other TAAR agonists and agents having similar properties and biological, physiological, or pharmacologic responses can be used. The examples, along with the methods and techniques disclosed herein provide a skilled artisan with the guidance to carry out similar studies with any other agent (or variant thereof) disclosed herein. For example, one skilled in the art will appreciate that an agent that excites the pilomotor reflex or a similar pilomotor response can be a candidate for use in any of the compositions and methods disclosed herein. One skilled in the art will further appreciate a human subject that experiences an excitation of the pilomotor reflex or a similar pilomotor response can be a candidate for being treated by any of the compositions and methods disclosed herein.

It should be noted that the examples disclosed herein (in particular Examples 8 and 9) pertain to compositions with tyramine HCl solutions at 5%, 10%, and 12%. Tests results for tyramine HCl solutions at 15% and 20% were similar, and in some cases the same. Compositions with tyramine HCl solutions at or above 15% may be more beneficial for compositions formulated as shampoos or lotions than compositions with tyramine HCl solutions below 15%.

Methods and Techniques that May Used:

Patients: Five female subjects, ages 18-40, were included in the study. Subjects were recruited based on their frequent use of traumatic hair care practices, such as, tight braids, head scarves, ponytails, extensions, hair rollers, hair weaves and heated styling appliances such as blow dryers, flat irons, heat setters and curling irons. Subjects with uncontrolled hypertension, that were pregnant or breastfeeding, had been diagnosed with pattern hair loss or with other hair loss in conjunction with female pattern hair loss were excluded from the study. Prior to initiating the study, the efficacy of the 12% tyramine solution was tested by applying a small aliquot (50 μL) of the solution to the forearm of three subjects. Piloerection and blanching were visible after 30 minutes; the effect lasted for approximately 2-3 hours.

Hair Shedding: To measure hair loss during cosmetic procedures, a 4-day protocol was designed. On the first day patients were instructed to wash their hair and use styling products and procedures as they normally would. On the second day, patients were instructed not to wash their hair and to apply 0.5 mL of a placebo solution, containing a vehicle only, on the frontal area of the scalp in an 8×10 cm² target area. Patients were instructed to wait 45 minutes, after which, they brushed their hair 20 times from the front of the scalp to the bottom of head using a new brush. After the procedure, the brushes were sealed in a plastic bag. On day three, patients were instructed to wash their hair and use styling products and procedures as they normally would. On the fourth day, patients repeated the procedures of day two; only they applied 0.5 mL of a 12% tyramine solution to the target area. After each clinical procedure, the investigator counted the hairs collected on each brush. A new brush was used for each procedure.

Epilation Force:

To evaluate the effect of a topically applied TAAR agonist combination on the force required to pluck hairs from the scalp, a hand-held spring dynamometer, or “trichotillometer” was used. The trichotillometer records the maximum force threshold, in grams, required to pluck a single hair from the scalp; the performance and statistical variance of the instrument have been reported previously. Force measurements were performed using the trichotillometer on 10 subjects. The frontal area of scalp was divided into two 8×10 cm² areas. On the right side 0.5 mL of a placebo vehicle was applied. On the left side, 0.5 mL a 12% tyramine solution was applied. After 45 minutes, ten hairs were plucked from each of the target areas with the trichotillometer.

### Results:

After tabulating the data of 15 subjects studied in the hair shedding experiment (Table 6), we found a decrease in hair loss in 4 out of 5 patients (80%) in the target area following the application of 12% tyramine solution compared to hair loss in the targeted area following the application of a placebo solution. Reduction in hair loss varied from 9% to 100%, with an average reduction of 49%.

TABLE 6
Number of hairs removed with brush after the application of 12%
tyramine or placebo.

| | Number of Hairs Removed with Brush | | |
|---|---|---|---|
| Patient # | Placebo | 12% Tyramine | Reduction |
| 1 | 15 | 13 | 13% |
| 2 | 24 | 6 | 75% |
| 3 | 9 | 9 | 0% |
| 4 | 8 | 4 | 50% |
| 5 | 22 | 11 | 50% |

Measurements of the epilation force threshold in 5 subjects showed similar improvements (Table 7). The epilation force threshold on scalp hair follicles increased 376% on average following the application of a topical 12% tyramine solution.

TABLE 7
Grams of force required for epilation after the application of 12%
tyramine or placebo. Each data point is the average of 10 plucked
hairs [avg. (std.)].

| | Epilation Force (grams) | | |
|---|---|---|---|
| Patient # | Placebo | 12% Tyramine | Increase |
| 1 | 8.4 (2.6) | 18.0 (6.6) | 214% |
| 2 | 16.7 (2.7) | 32.8 (2.7) | 196% |
| 3 | 9.3 (2.4) | 49.8 (4.6) | 535% |
| 4 | 5.1 (2.4) | 39.0 (7.9) | 764% |
| 5 | 7.3 (1.5) | 12.6 (4.8) | 172% |

### Example 10: TAAR Shampoo with Tyramine Hydrochloride

Approximately 40% of women experience excessive hair shedding when washing their hair. It has been previously demonstrated that a topically applied al adrenergic receptor agonist can be used to contract the arrector pili muscle of the follicular unit (i.e., produce “goose bumps”), increasing the force required to pluck hair by as much as 400%. It has also been previously demonstrated that a topical cosmetic solution containing an al adrenergic receptor agonist can be used to reduce hair shedding during brushing by a maximum of 77%. Embodiments disclosed herein relate to mechanisms to contract the arrector pili muscle using trace amine associated receptor agonists. Trace amine associated receptors (TAAR) have been shown to regulate smooth muscle tone in blood vessels, but have not been reported to be present in the skin. Yet, the inventors have discovered the anti-shedding efficacy of a shampoo containing a selective TAAR agonist, tyramine hydrochloride. A study was conducted with an embodiment of the TAAR agonist shampoo, and the results indicate that embodiments of the TAAR shampoo reduced hair shedding during brushing by 31% in a cohort of 24 women with a maximum reduction of 77%.

Hair shedding is a normal part of the physiological processes of the body. Normally, women shed between 50 to 150 hairs in a 24-h period, while shedding in excess of 150 hairs can indicate thinning. In a survey of 300 women visiting a public hospital for conditions other than alopecia, approximately 40% reported experiencing excessive hair shedding subsequent to hair washing (as defined by the Sinclair Hair Shedding Scale). Increased shedding in women can sometimes be attributed to underlying medical conditions, however, even in healthy individuals many common hair styling practices can lead to excess shedding. For example, blow drying, flat ironing, hair curling, and brushing all apply traumatic force to hair follicles and consequently remove hair.

As noted herein, hair follicles in the scalp are connected to the inner surface of the basal epidermis by smooth muscle, i.e., the arrector pili muscle. Its contraction produces piloerection, which is characterized by the phenotypical puckering of the skin around the hair or “goose bump”. Piloerection can be stimulated by signaling molecules binding to receptors present on the arrector pili muscle. The force applied to the hair shaft during contraction of the arrector pili can greatly increases the threshold of force required to pluck hair.

It is recognized that two molecules known to be agonists of the α1 adrenergic receptor (α1-AR), a G-protein coupled receptor, expressed on the arrector pili muscle. In a pilot study of 15 female subjects, the inventors demonstrated that topically applying the α1 adrenergic receptor agonist, phenylephrine hydrochloride, increased the average threshold of force required to pluck hair by 172%. Additionally, topical phenylephrine reduced shedding during brushing by 40%. In a follow up study, the inventors demonstrated a topical formula containing synephrine hydrochloride (AB-102) had similar efficacy. AB-102 reduced the number of hairs lost during brushing by 38% in a cohort of 40 women with a maximum reduction of 77%. Furthermore, the threshold force for epilation was increased by a maximum of 86%. During both studies, no cardiac or hemodynamic changes were observed in any of the subjects sampled.

As noted herein, TAARs are a different class of G protein-coupled receptors. TAARs bind trace amines found naturally in mammals, for example, phenylethylamine, tyramine, and tryptamine. Recently it has been reported that TAARs are located in vascular smooth muscle and can be stimulated to cause smooth muscle tone changes independent of the sympathetic mechanism, e.g., al-AR. The inventors hypothesized that a TAAR agonist could be used to induce piloerection and increase the threshold for epilation similar to the al-AR agonists previously studied.

The following study is a report on an embodiment of a TAAR agonist shampoo containing tyramine hydrochloride, a selective TAAR agonist. Twenty-four healthy female subjects, ages 18-65, were recruited from an outpatient dermatology clinic at Zagreb University Hospital. Subjects were recruited based on complaint of excessive hair shedding subsequent to hair washing. Subjects with uncontrolled hypertension, that were pregnant or breastfeeding, had been diagnosed with pattern hair loss or with other hair loss in conjunction with female pattern hair loss were excluded from the study. The study was conducted with the approval of the hospital ethics committee. All subjects gave informed consent during enrolment in the study.

To measure hair loss after hair washing, a 4-day protocol was designed. On the first day patients were instructed to wash their hair and use styling products as they normally would. On the second day, patients were instructed to wash their hair with a placebo shampoo, containing the vehicle only. Subsequent to hair washing, patients were instructed to brush their hair 20 times from the front of the scalp to the bottom of head using a new brush. After the procedure, the brushes were placed in a labeled plastic bag. On day three, patients were instructed to wash their hair and use styling products as they normally would. On the fourth day, patients repeated the procedures of day two; only they applied an embodiment of the TAAR agonist shampoo (a shampoo with 15% tyramine HCl (w/w)). At the end of the study, the investigator counted the hairs collected on each brush.

Twenty-four subjects completed the study. The raw data is tabulated in Table 8.

TABLE 8
Number of hairs removed by brushing after using
a shampoo containing tyramine versus placebo

| | Hairs Collected | |
|---|---|---|
| Patient # | Placebo | Tyramine |
| 1 | 3 | 12 |
| 2 | 9 | 9 |
| 3 | 8 | 14 |
| 4 | 11 | 8 |
| 5 | 16 | 11 |
| 6 | 8 | 7 |
| 7 | 42 | 17 |
| 8 | 5 | 11 |
| 9 | 44 | 36 |
| 10 | 26 | 16 |
| 11 | 66 | 39 |
| 12 | 18 | 5 |
| 13 | 31 | 12 |
| 14 | 20 | 2 |
| 15 | 10 | 6 |
| 16 | 8 | 4 |
| 17 | 20 | 4 |
| 18 | 54 | 26 |
| 19 | 85 | 82 |
| 20 | 22 | 5 |
| 21 | 62 | 57 |
| 22 | 22 | 7 |
| 23 | 55 | 58 |
| 24 | 25 | 17 |

The paired sample t-test was used to determine the effect of the TAAR agonist shampoo compared to the vehicle shampoo on hair shedding subsequent to hair washing. The mean hair shedding in the TAAR shampoo group was 19.38±20.58 compared to 27.92±22.36 in the placebo group. The average reduction in hairs lost during brushing after using the TAAR agonist shampoo versus placebo was 31% for the cohort with a maximum reduction of 77%. The analysis of the dataset (MedCalc v18.2.1) yielded a paired sample t-test value of 4.012 (p=0.005); indicating that the use of the TAAR agonist shampoo resulted in significant reduction in hair shedding subsequent to hair washing compared to the vehicle shampoo.

A significant amount of women report excessive shedding after washing their hair. To-date, no treatment is available for the reduction of hair shedding caused by the mechanical stress of everyday washing and styling. Previously, the inventors demonstrated that a solution containing synephrine hydrochloride significantly reduced hair shedding during brushing. A shampoo with synephrine hydrochloride may have some drawbacks due to the high concentration of the weak agonist required to affect the arrector pili muscles in hair follicles. This may not be the case with a TAAR agonist shampoo. The result of this study provides evidence of the expression of TAAR receptors in the arrector pili muscle. Additionally, embodiments of the TAAR agonist shampoo were demonstrated to contract the arrector pili muscle and reduce hair shedding subsequent to washing. The average reduction was 31% for the cohort with a maximum reduction of 77%.

Additional understanding of the underlying principles relied upon for the methods and techniques discloses herein can be appreciated from the following references, the contents of which are each incorporated herein by reference in its entirety.
1. Ozçelik D. Extensive traction alopecia attributable to ponytail hairstyle and its treatment with hair transplantation. Aesthetic Plast Surg 2005: 29(4): 325-327.
2. Hjorth N. Traumatic marginal alopecia; a special type: alopecia groenlandica. Br J Dermatol 1957: 69(9): 319-322.
3. Khumalo N P, Jessop S, Gumedze F, Ehrlich R. Determinants of marginal traction alopecia in African girls and women. J Am Acad Dermatol 2008: 59(3): 432-438.
4. Hellmann K. The isolated pilomotor muscles as an in vitro preparation. J Physiol 1963: 169: 603-620.
5. Siepmann T, Gibbons C H, Illigens B M, Lafo J A, Brown C M, Freeman R. Quantitative pilomotor axon reflex test: a novel test of pilomotor function. Arch Neurol 2012: 69(11): 1488-1492.
6. Lewis T, Marvin H M. Observations upon a pilomotor reaction in response to faradism. J Physiol 1927: 64(1): 87-106.
7. Piascik M T, Perez D M. Alphal-adrenergic receptors: new insights and directions. J Pharmacol Exp Ther 2001: 298(2): 403-410.
8. Wyness L A, McNeill G, Prescott G L. Trichotillometry: the reliability and practicality of hair pluckability as a method of nutritional assessment. Nutr J 2007: 6: 9.
9. Chase E S, Weinsier R L, Laven G T, Krumdieck C L. Trichotillometry: the quantitation of hair pluckability as a method of nutritional assessment. Am J Clin Nutr 1981: 34(10): 2280-2286.
10. Smelser D N, Smelser N B, Krumdieck C L, Schreeder M T, Laven G T. Field use of hair epilation force in nutrition status assessment. Am J Clin Nutr 1982: 35: 342-346.

1. A method for treatment or prevention of traction alopecia, reduction of hair shedding, causing contraction of an arrector pili muscle or increasing hair epilation force threshold, wherein the method comprises applying a composition comprising a pilomotor effective amount of a trace amine associated receptor agonist topically to a portion of skin that includes at least one hair follicle.

2. The method of claim 1, wherein the portion of skin that includes at least one hair follicle is on the head of a person.

3. The method of claim 1, wherein the at least one hair follicle is under tension.

4. The method of claim 1, wherein the portion of skin is at risk for developing traction alopecia.

5. The method of claim 1, wherein the composition comprises a trace amine associated receptor agonist that is octopamine, p-octopamine, m-octopamine, and/or a pharmaceutically acceptable salt or hydrate thereof, wherein the octopamine is present in the composition in a concentration of 1% to 60% by weight.

6. The method of claim 1, wherein the composition comprises at least one enantiomer of octopamine that is R-(−)-4-(2-amino-1-hydroxyethyl)phenol and has less than 10% by weight of other enantiomers of octopamine.

7. The method of claim 6, wherein R-(−)-4-(2-amino-1-hydroxyethyl)phenol is present in the composition at 5% to 40% by weight.

8. The method of claim 1, wherein the trace amine associated receptor agonist is any one or combination of citrus aurantium (e.g. bitter orange extract), 2-phenylethylamine, p-tyramine, m-tyramine, tyramine HCl, a pharmaceutically acceptable salt of tyramine, a hydrate of tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamylamine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO521017, RO5203648, RO5256390, RO5263397, and RO5212773 (EPPTB).

9. The method of claim 1, wherein the method further comprising reducing hair shedding during brushing, combing or showering after application of the composition.

10. The method of claim 1, wherein the method further comprises administering a therapeutically-effective concentration of a probiotic, a genetic modified bacteria, and/or a viral vector to cause local floral to generate a trace amine.

11. The method of claim 1, wherein the traction alopecia is frontal pattern traction alopecia.

12. A method for treatment or prevention of traction alopecia, reduction of hair shedding, causing contraction of an arrector pili muscle or increasing hair epilation force threshold, wherein the method comprises applying a composition comprising a pilomotor effective amount of a trace amine associated receptor agonist and an alpha 1 adrenergic receptor agonist topically to a portion of skin that includes at least one hair follicle.

13. The method of claim 12, wherein the portion of skin that includes at least one hair follicle is on the head of a person.

14. The method of claim 12, wherein the at least one hair follicle is under tension.

15. The method of claim 12, wherein the portion of skin is at risk for developing traction alopecia.

16. The method of claim 12, wherein the composition comprises a trace amine associated receptor agonist that is octopamine, p-octopamine, m-octopamine, and/or a pharmaceutically acceptable salt or hydrate thereof, wherein the octopamine is present in the composition in a concentration of 1% to 60% by weight.

17. The method of claim 12, wherein the composition comprises at least one enantiomer of octopamine that is R-(−)-4-(2-amino-1-hydroxyethyl)phenol and has less than 10% by weight of other enantiomers of octopamine.

18. The method of claim 17, wherein R-(−)-4-(2-amino-1-hydroxyethyl)phenol is present in the composition at 5% to 40% by weight.

19. The method of claim 12, wherein the trace amine associated receptor agonist is any one or combination of citrus aurantium (e.g. bitter orange extract), 2-phenylethylamine, p-tyramine, m-tyramine, tyramine HCl, a pharmaceutically acceptable salt of tyramine, a hydrate of tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamylamine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO521017, RO5203648, RO5256390, RO5263397, and RO5212773 (EPPTB); and
wherein the alpha 1 adrenergic receptor agonist is any one or combination of cirazoline, desvenlafaxine, etilfrine, metaraminol, methoxamine, naphazoline, oxymetazoline, pseudoephrine, m-synephrine, p-synephrine, synephrine, octopamine, hordenine, tetrahydrozoline, isometheptene, metaraminol, nicergoline, ergonovine, levonordefrin, phendimetrazine, methoxamine, midodrine, clonidine, pergolide, xylometazoline, droxidopa, epinephrine, mephentermine, 4-methoxyamphetamine, Benzphetamine, Naphazoline, Apraclondine, Bromocriptine, Oxymetazoline, Phenylpropanolamine, Pseudoephedrine, Dipivefrin, and xylometazoline.

20. The method of claim 12, wherein the method further comprising reducing hair shedding during brushing, combing or showering after application of the composition.

21. The method of claim 12, wherein the method further comprises administering a therapeutically-effective concentration of a probiotic, a genetic modified bacteria, and/or a viral vector to cause local floral to generate a trace amine.

22. The method of claim 12, wherein the traction alopecia is frontal pattern traction alopecia.

23. A method for treatment or prevention of traction alopecia, reduction of hair shedding, causing contraction of an arrector pili muscle or increasing hair epilation force threshold, wherein the method comprises applying a composition comprising a pilomotor effective amount of a trace amine associated receptor agonist and an alpha 1 adrenergic receptor antagonist topically to a portion of skin that includes at least one hair follicle.

24. The method of claim 23, wherein the portion of skin that includes at least one hair follicle is on the head of a person.

25. The method of claim 23, wherein the at least one hair follicle is under tension.

26. The method of claim 23, wherein the portion of skin is at risk for developing traction alopecia.

27. The method of claim 23, wherein the composition comprises a trace amine associated receptor agonist that is octopamine, p-octopamine, m-octopamine, and/or a pharmaceutically acceptable salt or hydrate thereof, wherein the octopamine is present in the composition in a concentration of 1% to 60% by weight.

28. The method of claim 23, wherein the composition comprises at least one enantiomer of octopamine that is R-(−)-4-(2-amino-1-hydroxyethyl)phenol and has less than 10% by weight of other enantiomers of octopamine.

29. The method of claim 28, wherein R-(−)-4-(2-amino-1-hydroxyethyl)phenol is present in the composition at 5% to 40% by weight.

30. The method of claim 23, wherein the trace amine associated receptor agonist is any one or combination of citrus aurantium (e.g. bitter orange extract), 2-phenylethylamine, p-tyramine, m-tyramine, tyramine HCl, a pharmaceutically acceptable salt of tyramine, a hydrate of tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamylamine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO521017, RO5203648, RO5256390, RO5263397, and RO5212773 (EPPTB); and
wherein the alpha 1 adrenergic receptor antagonist is any one or combination of (+)Dobutamine, abanoquil, Acebutolol, adimolol, ajmalicine, alfuzosin, anisodamine, Atenolol, benoxathian, Betaxolol, Bretylium, Buflomedil, Butoxamine, Carteolol, carvedilol, cirazoline, corynanthine, dihydroergocornine, dihydroergocristine, dihydroergocryptine, dihydroergotoxine, doxazosin, ergot derivatives, Esmolol, Guanadrel, Guanethidine, hydroxymaprotiline, ifenprodil, indoramin, ketanserin, labetalol, Levobunolol, Metoprolol, monatepil, Moxisylyte, Nadolol, nantenine, Nicergoline, oxaprotiline, pelanserin, Penbutolol, phendioxan, phenoxybenzamine, phentolamine, Pindolol, prazosin, Propanolol, pukateine, Raubasine, rauwolscine, Reserpine, silodosin, tamsulosin, terazosin, thiamenidine, tiamenidine, Timolol, Tolazoline, umespirone, urapidil, urapidil, WB-4101, yohimbine, ziprasidone, zuclopenthixol, L-765,314, Z-350, SR 59230A, and BMY-7,378.

31. The method of claim 23, wherein the method further comprising reducing hair shedding during brushing, combing or showering after application of the composition.

32. The method of claim 23, wherein the method further comprises administering a therapeutically-effective concentration of a probiotic, a genetic modified bacteria, and/or a viral vector to cause local floral to generate a trace amine.

33. The method of claim 23, wherein the traction alopecia is frontal pattern traction alopecia.

34. A composition comprising a pilomotor effective amount of a trace amine associated receptor agonist, a cosmetic hair product and optionally either an alpha 1 adrenergic receptor agonist or an alpha 1 adrenergic receptor antagonist.

35. The composition of claim 34, wherein the hair product comprises pre-shampoo tonic, shampoo, hair color, hair dye, hair oil, hair conditioner, hairspray, and/or hair detangling solution.

36. The composition of claim 34, wherein the trace amine associated receptor agonist is tyramine, tyramine HCl, and/or a pharmaceutically acceptable salt or hydrate thereof.

37. The composition of claim 36, wherein the tyramine, tyramine HCl, and/or a pharmaceutically acceptable salt or hydrate thereof is present in the composition in a concentration of 5% to 20% by weight.

38. The composition of claim 36, wherein the tyramine, tyramine HCl, and/or a pharmaceutically acceptable salt or hydrate thereof is present in the composition in a concentration of 1% to 10% by weight.

39. The composition of claim 36, wherein the tyramine, tyramine HCl, and/or a pharmaceutically acceptable salt or hydrate thereof is present in the composition in a concentration of 10% to 30% by weight.

40. The composition of claim 36, wherein the composition comprises 15% tyramine HCl solution to 20% tyramine HCl solution by weight.

41. The composition of claim 34, wherein the composition is configured as a pre-shampoo tonic, shampoo, hair color, hair dye, hair oil, hair conditioner, hairspray, hair detangling solution, moisturizing lotion, a facial cream, a sunscreen, a gel, an ointment, a foam, and/or a spray.

42. The composition of claim 34, wherein the trace amine associated receptor agonist is any one or combination of citrus aurantium (e.g. bitter orange extract), 2-phenylethylamine, p-tyramine, m-tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamylamine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO521017, RO5203648, RO5256390, RO5263397, and RO5212773 (EPPTB).

43. The composition of claim 34, wherein the composition comprises an alpha 1 adrenergic receptor agonist,
wherein the alpha 1 adrenergic receptor agonist is any one or combination of cirazoline, desvenlafaxine, etilfrine, metaraminol, methoxamine, naphazoline, oxymetazoline, pseudoephrine, m-synephrine, p-synephrine, synephrine, octopamine, hordenine, tetrahydrozoline, isometheptene, metaraminol, nicergoline, ergonovine, levonordefrin, phendimetrazine, methoxamine, midodrine, clonidine, pergolide, xylometazoline, droxidopa, epinephrine, mephentermine, 4-methoxyamphetamine, Benzphetamine, Naphazoline, Apraclondine, Bromocriptine, Oxymetazoline, Phenylpropanolamine, Pseudoephedrine, Dipivefrin, and xylometazoline; and
wherein the trace amine associated receptor agonist is any one or combination of citrus aurantium (e.g. bitter orange extract), 2-phenylethylamine, p-tyramine, m-tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamylamine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO521017, RO5203648, RO5256390, RO5263397, and RO5212773 (EPPTB).

44. The composition of claim 34, wherein the composition comprises an alpha 1 adrenergic receptor antagonist,
wherein the alpha 1 adrenergic receptor antagonist is any one or combination of (+)Dobutamine, abanoquil, Acebutolol, adimolol, ajmalicine, alfuzosin, anisodamine, Atenolol, benoxathian, Betaxolol, Bretylium, Buflomedil, Butoxamine, Carteolol, carvedilol, cirazoline, corynanthine, dihydroergocornine, dihydroergocristine, dihydroergocryptine, dihydroergotoxine, doxazosin, ergot derivatives, Esmolol, Guanadrel, Guanethidine, hydroxymaprotiline, ifenprodil, indoramin, ketanserin, labetalol, Levobunolol, Metoprolol, monatepil, Moxisylyte, Nadolol, nantenine, Nicergoline, oxaprotiline, pelanserin, Penbutolol, phendioxan, phenoxybenzamine, phentolamine, Pindolol, prazosin, Propanolol, pukateine, Raubasine, rauwolscine, Reserpine, silodosin, tamsulosin, terazosin, thiamenidine, tiamenidine, Timolol, Tolazoline, umespirone, urapidil, urapidil, WB-4101, yohimbine, ziprasidone, zuclopenthixol, L-765,314, Z-350, SR 59230A, and BMY-7,378; and
wherein the trace amine associated receptor agonist is any one or combination of citrus aurantium (e.g. bitter orange extract), 2-phenylethylamine, p-tyramine, m-tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamylamine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO521017, RO5203648, RO5256390, RO5263397, and RO5212773 (EPPTB).

45. A cosmetic kit comprising a container including the composition of claim 34.

46. The cosmetic kit of claim 45, wherein the container is a pump spray bottle.
